SEQUENCE LISTING (81 sequences)

SEQ ID NO: 1 - DNA, 144 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggt

SEQ ID NO: 2 - DNA, 150 bp, Artificial Sequence
  Synthesized oligonucleotide
tcgtatgaattcgcggccgcttctagagccacaattcagcaaattgtgaacatcatctccctggttgctcctgtcagtaagtaatgagatcggaagagcacacgtctgaactccagtcaccagatcatctcgtatgccgtcttctgcttg

SEQ ID NO: 3 - DNA, 178 bp, Artificial Sequence
  Synthesized oligonucleotide
ctgcgtctgagaggtggtacatgggtgaacttacttgcataccaagttgatacttgaataaccatctgaaagtggtacttgatcattttacatgggtgaacttacttgcataccaagttgatacttgaataaccatctgaaagtggtacttgatcatttttcgtatgaattcgcggcc

SEQ ID NO: 4 - DNA, 191 bp, Artificial Sequence
  Synthesized oligonucleotide
ctgcgtctgagaggtggtcatcaccatcaccatcaccatcaccaccatcattagatgaatatgaaacattttcacttgttcttcctactcacgcttctgtttcttacacccaggattcaggcacatcatcaccatcaccatcaccatcaccaccatcattagatgaatatgaatcgtatgaattcgcggcc

SEQ ID NO: 5 - DNA, 188 bp, Artificial Sequence
  Synthesized oligonucleotide
ctgcgtctgagaggtggtcaaggcataaaaccaaatctcattctctttcttctctattctttgcagccatgggtaattaccaacaacaacaaacaacaaacaacattacaattaataaaaccaaatctcattctctttcttctctattctttgcagccatgggtctgcagtcgtatgaattcgcggcc

SEQ ID NO: 6 - DNA, 174 bp, Artificial Sequence
  Synthesized oligonucleotide
ctgcgtctgagaggtggttattgcatacccgtttttaataaaatacattgcataccctcttttaataaaaaatattgcatactttgacgaaatattgcatacccgtttttaataaaatacattgcataccctcttttaataaaaaatattgcatactcgtatgaattcgcggcc

SEQ ID NO: 7 - DNA, 200 bp, Artificial Sequence
  Synthesized oligonucleotide
ctgcgtctgagaggtggtacgaaccagaggatccctgctagccaatggggcgatcgcccacaattgcggtggcggaaaatttaaaggatctggagggggcatcatcaggatccctgctagccaatggggcgatcgcccacaattgcggtggcggaaaatttaaaggatctggtgggggaggttcgtatgaattcgcggcc

SEQ ID NO: 8 - DNA, 85 bp, Artificial Sequence
  Synthesized oligonucleotide
ctgcgtctgagaggtggttcatccgcgagaccacacgccatcatcatcacgtgaagatgatatcgtttcgtatgaattcgcggcc

SEQ ID NO: 9 - DNA, 94 bp, Artificial Sequence
  Synthesized oligonucleotide
ctgcgtctgagaggtggttcatccgcgagaccacacgccatcatcatcatcatcatcacgtgaagatgatatcgtttcgtatgaattcgcggcc

SEQ ID NO: 10 - DNA, 103 bp, Artificial Sequence
  Synthesized oligonucleotide
ctgcgtctgagaggtggttcatccgcgagaccacacgccatcatcatcatcatcatcatcatcatcacgtgaagatgatatcgtttcgtatgaattcgcggcc

SEQ ID NO: 11 - DNA, 112 bp, Artificial Sequence
  Synthesized oligonucleotide
ctgcgtctgagaggtggttcatccgcgagaccacacgccatcatcatcatcatcatcatcatcatcatcatcatcacgtgaagatgatatcgtttcgtatgaattcgcggcc

SEQ ID NO: 12 - DNA, 121 bp, Artificial Sequence
  Synthesized oligonucleotide
ctgcgtctgagaggtggttcatccgcgagaccacacgccatcatcatcatcatcatcatcatcatcatcatcatcatcatcatcacgtgaagatgatatcgtttcgtatgaattcgcggcc

SEQ ID NO: 13 - DNA, 130 bp, Artificial Sequence
  Synthesized oligonucleotide
ctgcgtctgagaggtggttcatccgcgagaccacacgccatcatcatcatcatcatcatcatcatcatcatcatcatcatcatcatcatcatcacgtgaagatgatatcgtttcgtatgaattcgcggcc

SEQ ID NO: 14 - DNA, 81 bp, Artificial Sequence
  Synthesized oligonucleotide
ctgcgtctgagaggtggttcatccgcgagaccacacgcgggggcacgtgaagatgatatcgtttcgtatgaattcgcggcc

SEQ ID NO: 15 - DNA, 82 bp, Artificial Sequence
  Synthesized oligonucleotide
ctgcgtctgagaggtggttcatccgcgagaccacacgcggggggcacgtgaagatgatatcgtttcgtatgaattcgcggcc

SEQ ID NO: 16 - DNA, 83 bp, Artificial Sequence
  Synthesized oligonucleotide
ctgcgtctgagaggtggttcatccgcgagaccacacgcgggggggcacgtgaagatgatatcgtttcgtatgaattcgcggcc

SEQ ID NO: 17 - DNA, 84 bp, Artificial Sequence
  Synthesized oligonucleotide
ctgcgtctgagaggtggttcatccgcgagaccacacgcggggggggcacgtgaagatgatatcgtttcgtatgaattcgcggcc

SEQ ID NO: 18 - DNA, 85 bp, Artificial Sequence
  Synthesized oligonucleotide
ctgcgtctgagaggtggttcatccgcgagaccacacgcgggggggggcacgtgaagatgatatcgtttcgtatgaattcgcggcc

SEQ ID NO: 19 - DNA, 86 bp, Artificial Sequence
  Synthesized oligonucleotide
ctgcgtctgagaggtggttcatccgcgagaccacacgcggggggggggcacgtgaagatgatatcgtttcgtatgaattcgcggcc

SEQ ID NO: 20 - DNA, 19 bp, Artificial Sequence
  Synthesized oligonucleotide
aatgatacggcgaccaccg

SEQ ID NO: 21 - DNA, 21 bp, Artificial Sequence
  Synthesized oligonucleotide
caagcagaagacggcatacga

SEQ ID NO: 22 - DNA, 436 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggtacatgggtgaacttacttgcataccaagttgatacttgaataaccatctgaaagtggtacttgatcattttacatgggtgaacttacttgcataccaagttgatacttgaataaccatctgaaagtggtacttgatcatttttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 23 - DNA, 449 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggtcatcaccatcaccatcaccatcaccaccatcattagatgaatatgaaacattttcacttgttcttcctactcacgcttctgtttcttacacccaggattcaggcacatcatcaccatcaccatcaccatcaccaccatcattagatgaatatgaatcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 24 - DNA, 446 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggtcaaggcataaaaccaaatctcattctctttcttctctattctttgcagccatgggtaattaccaacaacaacaaacaacaaacaacattacaattaataaaaccaaatctcattctctttcttctctattctttgcagccatgggtctgcagtcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 25 - DNA, 432 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttattgcatacccgtttttaataaaatacattgcataccctcttttaataaaaaatattgcatactttgacgaaatattgcatacccgtttttaataaaatacattgcataccctcttttaataaaaaatattgcatactcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 26 - DNA, 458 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggtacgaaccagaggatccctgctagccaatggggcgatcgcccacaattgcggtggcggaaaatttaaaggatctggagggggcatcatcaggatccctgctagccaatggggcgatcgcccacaattgcggtggcggaaaatttaaaggatctggtgggggaggttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 27 - DNA, 343 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgccatcatcatcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 28 - DNA, 352 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgccatcatcatcatcatcatcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 29 - DNA, 361 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgccatcatcatcatcatcatcatcatcatcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 30 - DNA, 370 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgccatcatcatcatcatcatcatcatcatcatcatcatcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 31 - DNA, 379 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgccatcatcatcatcatcatcatcatcatcatcatcatcatcatcatcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 32 - DNA, 388 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgccatcatcatcatcatcatcatcatcatcatcatcatcatcatcatcatcatcatcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 33 - DNA, 339 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgcgggggcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 34 - DNA, 340 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgcggggggcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 35 - DNA, 341 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgcgggggggcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 36 - DNA, 342 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgcggggggggcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 37 - DNA, 343 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgcgggggggggcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 38 - DNA, 344 bp, Artificial Sequence
  Synthesized oligonucleotide
aatgatacggcgaccaccgagatctacactctttccctacacgacgctcttccgatctgctagcgccggatcttcgtgacaagaccatcaccacttgacagttggccgtcgaccctgcacctggtcctgcgtctgagaggtggttcatccgcgagaccacacgcggggggggggcacgtgaagatgatatcgtttcgtatgaattcgcggccgcttctagagccacaattcagcaaattgtgaacatcatctccctggttgctcctgtcagtaagtaatgagatcggaagagcacacgtctgaactccagtcaccgatgtatctcgtatgccgtcttctgcttg

SEQ ID NO: 39 - DNA, 173 bp, Artificial Sequence
  Synthesized oligonucleotide
aatgatacggcgaccaccgagatctacactctttccctacacgacgctcttccgatcttacgactcactatagggagacccaagctggctagcgccggatcttcgtgacaagaccatcaccacttgacagttggccgtcgaccctgcacctggtcctgcgtctgagaggtggt

SEQ ID NO: 40 - DNA, 179 bp, Artificial Sequence
  Synthesized oligonucleotide
tcgtatgaattcgcggccgcttctagagccacaattcagcaaattgtgaacatcatctccctggttgctcctgtcagtaagtaatgaatactagtagcggccgctgcaggctaacagatcggaagagcacacgtctgaactccagtcaccgatgtatctcgtatgccgtcttctgcttg

SEQ ID NO: 41 - DNA, 39 bp, Artificial Sequence
  Synthesized oligonucleotide
ctgcgtctgagaggtggtnnktcgtatgaattcgcggcc

SEQ ID NO: 42 - DNA, 19 bp, Artificial Sequence
  Synthesized oligonucleotide
aatgatacggcgaccaccg

SEQ ID NO: 43 - DNA, 21 bp, Artificial Sequence
  Synthesized oligonucleotide
caagcagaagacggcatacga

SEQ ID NO: 44 - DNA, 355 bp, Artificial Sequence
  Synthesized oligonucleotide
aatgatacggcgaccaccgagatctacactctttccctacacgacgctcttccgatcttacgactcactatagggagacccaagctggctagcgccggatcttcgtgacaagaccatcaccacttgacagttggccgtcgaccctgcacctggtcctgcgtctgagaggtggtnnktcgtatgaattcgcggccgcttctagagccacaattcagcaaattgtgaacatcatctccctggttgctcctgtcagtaagtaatgaatactagtagcggccgctgcaggctaacagatcggaagagcacacgtctgaactccagtcaccgatgtatctcgtatgccgtcttctgcttg

SEQ ID NO: 45 - DNA, 179 bp, Artificial Sequence
  Synthesized oligonucleotide
tcgtatgaattcgcggccgcttctagagccacaattcagcaaattgtgaacatcatctccctggttgctcctgtcagtaagtaatgaatactagtagcggccgctgcaggctaacagatcggaagagcacacgtctgaactccagtcacgatcagatctcgtatgccgtcttctgcttg

SEQ ID NO: 46 - DNA, 54 bp, Artificial Sequence
  Synthesized oligonucleotide
ctgcgtctgagaggtggtnnknnknnknnknnknnktcgtatgaattcgcggcc

SEQ ID NO: 47 - DNA, 370 bp, Artificial Sequence
  Synthesized oligonucleotide
aatgatacggcgaccaccgagatctacactctttccctacacgacgctcttccgatcttacgactcactatagggagacccaagctggctagcgccggatcttcgtgacaagaccatcaccacttgacagttggccgtcgaccctgcacctggtcctgcgtctgagaggtggtnnknnknnknnknnknnktcgtatgaattcgcggccgcttctagagccacaattcagcaaattgtgaacatcatctccctggttgctcctgtcagtaagtaatgaatactagtagcggccgctgcaggctaacagatcggaagagcacacgtctgaactccagtcacgatcagatctcgtatgccgtcttctgcttg

SEQ ID NO: 48 - DNA, 224 bp, Artificial Sequence
  Synthesized oligonucleotide
tgctgctcctcgctgcccagccggcgatggccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccacc

SEQ ID NO: 49 - DNA, 193 bp, Artificial Sequence
  Synthesized oligonucleotide
cgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcc

SEQ ID NO: 50 - DNA, 63 bp, Artificial Sequence
  Synthesized oligonucleotide
cccaccctcgtgaccaccnnknnktacggcnnkcagtgcttcnnkcgctaccccgaccacatg

SEQ ID NO: 51 - DNA, 16 bp, Artificial Sequence
  Synthesized oligonucleotide
tgctgctcctcgctgc

SEQ ID NO: 52 - DNA, 20 bp, Artificial Sequence
  Synthesized oligonucleotide
ggatgttgccgtcctccttg

SEQ ID NO: 53 - DNA, 444 bp, Artificial Sequence
  Synthesized oligonucleotide
tgctgctcctcgctgcccagccggcgatggccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccnnknnktacggcnnkcagtgcttcnnkcgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcc

SEQ ID NO: 54 - DNA, 226 bp, Artificial Sequence
  Synthesized oligonucleotide
gcggagggtcggctagcggtcaagttcagttggttcaatcaggtgcggaagttaaaaagcctggtgcttctgttaaggtttcttgtaaagcctctggctatacttttacgggttattacatgcattgggtaagacaggctcccggtcagggtttggaatggatgggttggattaacccaaactctggtggaactaactatgctcaaaaattccaaggtagagttac

SEQ ID NO: 55 - DNA, 150 bp, Artificial Sequence
  Synthesized oligonucleotide
ttgtcacgtttgaggtctgatgatactgctgtttattactgtgctagaggtaagaactctgattacaattgggatttccaacattggggccagggcactttggttactgtttcaagtggtggtggaggatccggcggtggtgtcgtacgg

SEQ ID NO: 56 - DNA, 88 bp, Artificial Sequence
  Synthesized oligonucleotide
gctcaaaaattccaaggtagagttaccatgnnkagggatacttctatatctactgcttatatggaattgtcacgtttgaggtctgatg

SEQ ID NO: 57 - DNA, 88 bp, Artificial Sequence
  Synthesized oligonucleotide
gctcaaaaattccaaggtagagttactatgacannkgacacttctatatctactgcttatatggaattgtcacgtttgaggtctgatg

SEQ ID NO: 58 - DNA, 88 bp, Artificial Sequence
  Synthesized oligonucleotide
gctcaaaaattccaaggtagagttactatgactaggnnkacatctatatctactgcttatatggaattgtcacgtttgaggtctgatg

SEQ ID NO: 59 - DNA, 88 bp, Artificial Sequence
  Synthesized oligonucleotide
gctcaaaaattccaaggtagagttactatgactagagacnnktcaatatctactgcttatatggaattgtcacgtttgaggtctgatg

SEQ ID NO: 60 - DNA, 88 bp, Artificial Sequence
  Synthesized oligonucleotide
gctcaaaaattccaaggtagagttactatgactagagatacannkatttctactgcttatatggaattgtcacgtttgaggtctgatg

SEQ ID NO: 61 - DNA, 88 bp, Artificial Sequence
  Synthesized oligonucleotide
gctcaaaaattccaaggtagagttactatgactagagatacttcannktcaactgcttatatggaattgtcacgtttgaggtctgatg

SEQ ID NO: 62 - DNA, 88 bp, Artificial Sequence
  Synthesized oligonucleotide
gctcaaaaattccaaggtagagttactatgactagagatacttctattnnkacagcttatatggaattgtcacgtttgaggtctgatg

SEQ ID NO: 63 - DNA, 88 bp, Artificial Sequence
  Synthesized oligonucleotide
gctcaaaaattccaaggtagagttactatgactagagatacttctatatcannkgcatatatggaattgtcacgtttgaggtctgatg

SEQ ID NO: 64 - DNA, 88 bp, Artificial Sequence
  Synthesized oligonucleotide
gctcaaaaattccaaggtagagttactatgactagagatacttctatatctacannktacatggaattgtcacgtttgaggtctgatg

SEQ ID NO: 65 - DNA, 88 bp, Artificial Sequence
  Synthesized oligonucleotide
gctcaaaaattccaaggtagagttactatgactagagatacttctatatctactgcannkatggagttgtcacgtttgaggtctgatg

SEQ ID NO: 66 - DNA, 16 bp, Artificial Sequence
  Synthesized oligonucleotide
gcggagggtcggctag

SEQ ID NO: 67 - DNA, 16 bp, Artificial Sequence
  Synthesized oligonucleotide
caccaccgccggatcc

SEQ ID NO: 68 - DNA, 17 bp, Artificial Sequence
  Synthesized oligonucleotide
gccttgccagcccgctc

SEQ ID NO: 69 - DNA, 17 bp, Artificial Sequence
  Synthesized oligonucleotide
gcctccctcgcgccatc

SEQ ID NO: 70 - DNA, 331 bp, Artificial Sequence
  Synthesized oligonucleotide
gccttgccagcccgctcaggcataacttggacatgccaacttggaagggagaacgaagtcagtcatcaggcagactgggtcatctgctgaaatcacttgtgatcttgctgaaggaagtaacggctacatccactggtacctacaccaggaggggaaggccccacagcgtcttcagtactatgactcctacaactccaaggttgtgttggaatcaggagtcagtccagggaagtattatacttacgcaagcacaaggaacaacttgagattgatactgcgaaatctaattgaaaatgactttggggtctattactgtgccacctgggtcgac

SEQ ID NO: 71 - DNA, 151 bp, Artificial Sequence
  Synthesized oligonucleotide
gcataacttggacatgagtgattggatcaagacgtttgcaaaagggactaggctcatagtaacttcgcctggtaagtaattttttttctgtttttattccagtaatgaaaaactgagcataacttggacatgctgatggcgcgagggaggc

SEQ ID NO: 72 - DNA, 56 bp, Artificial Sequence
  Synthesized oligonucleotide
ctgtgccacctgggtcgacnnnnnnnnnnnngcataacttggacatgagtgattgg

SEQ ID NO: 73 - DNA, 494 bp, Artificial Sequence
  Synthesized oligonucleotide
gccttgccagcccgctcaggcataacttggacatgccaacttggaagggagaacgaagtcagtcatcaggcagactgggtcatctgctgaaatcacttgtgatcttgctgaaggaagtaacggctacatccactggtacctacaccaggaggggaaggccccacagcgtcttcagtactatgactcctacaactccaaggttgtgttggaatcaggagtcagtccagggaagtattatacttacgcaagcacaaggaacaacttgagattgatactgcgaaatctaattgaaaatgactttggggtctattactgtgccacctgggtcgacnnnnnnnnnnnngcataacttggacatgagtgattggatcaagacgtttgcaaaagggactaggctcatagtaacttcgcctggtaagtaattttttttctgtttttattccagtaatgaaaaactgagcataacttggacatgctgatggcgcgagggaggc

SEQ ID NO: 74 - DNA, 331 bp, Artificial Sequence
  Synthesized oligonucleotide
gccttgccagcccgctcagacgtactctggacatgtagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggtcgac

SEQ ID NO: 75 - DNA, 151 bp, Artificial Sequence
  Synthesized oligonucleotide
acgtactctggacatgagtgattggatcaagacgtttgcaaaagggactaggctcatagtaacttcgcctggtaagtaattttttttctgtttttattccagtaatgaaaaactgaacgtactctggacatgctgatggcgcgagggaggc

SEQ ID NO: 76 - DNA, 51 bp, Artificial Sequence
  Synthesized oligonucleotide
ctgtgccttgtgggtcgacnnnnnnnnnnnnacgtactctggacatgagtg

SEQ ID NO: 77 - DNA, 494 bp, Artificial Sequence
  Synthesized oligonucleotide
gccttgccagcccgctcagacgtactctggacatgtagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggtcgacnnnnnnnnnnnnacgtactctggacatgagtgattggatcaagacgtttgcaaaagggactaggctcatagtaacttcgcctggtaagtaattttttttctgtttttattccagtaatgaaaaactgaacgtactctggacatgctgatggcgcgagggaggc

SEQ ID NO: 78 - DNA, 331 bp, Artificial Sequence
  Synthesized oligonucleotide
gccttgccagcccgctcagcttctaagtggacatgtggagcagttccagctatccatttccacggaagtcaagaaaagtattgacataccttgcaagatatcgagcacaaggtttgaaacagatgtcattcactggtaccggcagaaaccaaatcaggctttggagcacctgatctatattgtctcaacaaaatccgcagctcgacgcagcatgggtaagacaagcaacaaagtggaggcaagaaagaattctcaaactctcacttcaatccttaccatcaagtccgtagagaaagaagacatggccgtttactactgtgctgcggtcgac

SEQ ID NO: 79 - DNA, 151 bp, Artificial Sequence
  Synthesized oligonucleotide
cttctaagtggacatgagtgattggatcaagacgtttgcaaaagggactaggctcatagtaacttcgcctggtaagtaattttttttctgtttttattccagtaatgaaaaactgacttctaagtggacatgctgatggcgcgagggaggc

SEQ ID NO: 80 - DNA, 53 bp, Artificial Sequence
  Synthesized oligonucleotide
ctgtgctgcggtcgacnnnnnnnnnnnncttctaagtggacatgagtgattgg

SEQ ID NO: 81 - DNA, 494 bp, Artificial Sequence
  Synthesized oligonucleotide
gccttgccagcccgctcagcttctaagtggacatgtggagcagttccagctatccatttccacggaagtcaagaaaagtattgacataccttgcaagatatcgagcacaaggtttgaaacagatgtcattcactggtaccggcagaaaccaaatcaggctttggagcacctgatctatattgtctcaacaaaatccgcagctcgacgcagcatgggtaagacaagcaacaaagtggaggcaagaaagaattctcaaactctcacttcaatccttaccatcaagtccgtagagaaagaagacatggccgtttactactgtgctgcggtcgacnnnnnnnnnnnncttctaagtggacatgagtgattggatcaagacgtttgcaaaagggactaggctcatagtaacttcgcctggtaagtaattttttttctgtttttattccagtaatgaaaaactgacttctaagtggacatgctgatggcgcgagggaggc

(19) United States
(12) Patent Application Publication
(10) Pub. No.: US 2015/0159152 A1
(43) Pub. Date: Jun. 11, 2015

(54) LONG NUCLEIC ACID SEQUENCES CONTAINING VARIABLE REGIONS

(71) Applicant: INTEGRATED DNA TECHNOLOGIES, INC., Skokie, IL (US)

(72) Inventors: Shawn Allen, Williamsburg, IA (US); Kristin Beltz, Cedar Rapids, IA (US); Scott Rose, Coralville, IA (US)

(73) Assignee: INTEGRATED DNA TECHNOLOGIES, INC., Skokie, IL (US)

(21) Appl. No.: 14/564,504

(22) Filed: Dec. 9, 2014

Related U.S. Application Data
(60) Provisional application No. 61/913,688, filed on Dec. 9, 2013.

(51) Int. Cl. C12N 15/10

(52) U.S. Cl. CPC C12N 15/1031; C12N 15/1068

(57) ABSTRACT

This invention pertains to improved methods for the synthesis of long, double stranded nucleic acid sequences containing difficult to clone or variable regions.

### CROSS-REFERENCE TO RELATED APPLICATIONS

This patent application claims priority to U.S. Provisional Patent Application No. 61/913,688 filed Dec. 9, 2013, the content of which is incorporated herein by reference in its entirety.

### SEQUENCE LISTING

The sequence listing is filed with the application in electronic format only and is incorporated by reference herein. The sequence listing text file “vBlock Sequence List” was created on Dec. 9, 2014 and is 33 kb in size.

### FIELD OF THE INVENTION

This invention pertains to improved methods for the synthesis of long, double stranded nucleic acid sequences containing regions of low complexity, repeating elements, difficult to assemble and clone elements, or variable regions containing mixed bases.

### BACKGROUND OF THE INVENTION

Synthetic DNA sequences are a vital tool in molecular biology. They are used in gene therapy, vaccines, DNA libraries, environmental engineering, diagnostics, tissue engineering and research into genetic variants. Long artificially-made nucleic acid sequences are commonly referred to as synthetic genes; however the artificial elements produced do not have to encode for genes, but, for example, can be regulatory or structural elements. Regardless of functional usage, long artificially-assembled nucleic acids can be referred to herein as synthetic genes and the process of manufacturing these species can be referred to as gene synthesis. Gene synthesis provides an advantageous alternative from obtaining genetic elements through traditional means, such as isolation from a genomic DNA library, isolation from a cDNA library, or PCR cloning. Traditional cloning requires availability of a suitable library constructed from isolated natural nucleic acids wherein the abundance of the gene element of interest is at a level that assures a successful isolation and recovery.

Artificial gene synthesis can also provide a DNA sequence that is codon optimized. Given codon redundancy, many different DNA sequences can encode the same amino acid sequence. Codon preferences differ between organisms and a gene sequence that is expressed well in one organism might be expressed poorly or not at all when introduced into a different organism. The efficiency of expression can be adjusted by changing the nucleotide sequence so that the element is well expressed in whatever organism is desired, e.g., it is adjusted for the codon bias of that organism. Widespread changes of this kind are easily made using gene synthesis methods but are not feasible using site-directed mutagenesis or other methods which introduce alterations into naturally isolated nucleic acids.

As another example, a synthetic gene can have restriction sites removed and new sites added. As yet another example, a synthetic gene can have novel regulatory elements or processing signals included which are not present in the native gene. Many other examples of the utility of gene synthesis are well known to those with skill in the art.

Furthermore, a sequence isolated from genomic DNA or cDNA libraries only provides an isolate having that nucleic acid sequence as it exists in nature. It is often desirable to introduce alterations into that sequence. For example a randomized mutant library can be created wherein random bases are inserted into desired positions and then expressed to find desirable properties relative to the wild type sequence. This approach does not allow for specific placement of degenerate bases. In another example, a gene enriched with repeat sequences could be used for genomic mapping or marking.

Although the cost of synthesizing a large library of genes can be substantial, the ability to optimize or change the characteristics of the encoded enzyme or antibody can result in a powerful biological tool or therapeutic. Recombinant antibodies such as Humira® (Abbot Laboratories, Inc.) are widely used as therapeutics, and many others are used as research tools. Those in the art also appreciate that many commercial proteins, such as enzymes, originated from mutant libraries.

Gene synthesis employs synthetic oligonucleotides as the primary building block. Oligonucleotides are made using chemical synthesis, most commonly using betacyanoethyl phosphoramidite methods, which are well-known to those with skill in the art (M. H. Caruthers, Methods in Enzymology 154, 287-313 (1987)). Using a four-step process, phosphoramidite monomers are added in a 3′ to 5′ direction to form an oligonucleotide chain. During each cycle of monomer addition, a small amount of oligonucleotides will fail to couple (n−1 product). Therefore, with each subsequent monomer addition the cumulative population of failures grows. Also, as the oligonucleotide grows longer, the base addition chemistry becomes less efficient, presumably due to steric issues with chain folding. Typically, oligonucleotide synthesis proceeds with a base coupling efficiency of around 99.0 to 99.2%. A 20 base long oligonucleotide requires 19 base coupling steps. Thus assuming a 99% coupling efficiency, a 20 base oligonucleotide should have 0.99¹⁹ purity, meaning approximately 82% of the final end product will be full length and 18% will be truncated failure products. A 40 base oligonucleotide should have 0.99³⁹ purity, meaning approximately 68% of the final end product will be full length and 32% will be truncated failure products. A 100 base oligonucleotide should have 0.99⁹⁹ purity, meaning approximately 37% of the final product will be full length and 63% will be truncated failure products. In contrast, if the efficiency of base coupling is increased to 99.5%, then a 100 base oligonucleotide should have a 0.995⁹⁹ purity, meaning approximately 61% of the final product will be full length and 39% will be truncated failure products.

Using gene synthesis methods, a series of synthetic oligonucleotides are assembled into a longer synthetic nucleic acid, e.g. a synthetic gene. The use of synthetic oligonucleotide building blocks in gene synthesis methods with a high percentage of failure products present will decrease the quality of the final product, requiring implementation of costly and time-consuming error correction methods. For this reason, relatively short synthetic oligonucleotides in the 40-60 base length range have typically been employed in gene synthesis methods, even though longer oligonucleotides could have significant benefits in assembly. It is well appreciated by those with skill in the art that use of high quality synthetic oligonucleotides, e.g. oligonucleotides with few error or missing bases, will result in high quality assembly of synthetic genes than the use of lower quality synthetic oligonucleotides.

Some common forms of gene assembly are ligation-based assembly, PCR-driven assembly (see Tian et al., Mol. BioSyst., 5, 714-722 (2009)) and thermodynamically balanced inside-out based PCR (TBIO) (see Gao X. et al., Nucleic Acids Res. 31, e143). All three methods combine multiple shorter oligonucleotides into a single longer end-product.

Therefore, to make genes that are typically 500 to many thousands of bases long, a large number of smaller oligonucleotides are synthesized and combined through ligation, overlapping, etc., after synthesis. Typically, gene synthesis methods only function well when combining a limited number of synthetic oligonucleotide building blocks and very large genes must be constructed from smaller subunits using iterative methods. For example, 10-20 of 40-60 base overlapping oligonucleotides are assembled into a single 500 base subunit due to the need for overlapping ends, and twelve or more 500 base overlapping subunits are assembled into a single 5000 base synthetic gene. Each subunit of this process is typically cloned (i.e., ligated into a plasmid vector, transformed into a bacterium, expanded, and purified) and its DNA sequence is verified before proceeding to the next step. If the above gene synthesis process has low fidelity, either due to errors introduced by low quality of the initial oligonucleotide building blocks or during the enzymatic steps of subunit assembly, then increasing numbers of cloned isolates must be sequence verified to find a perfect clone to move forward in the process or an error-containing clone must have the error corrected using site directed mutagenesis.

Traditional methods for assembly have suffered from shortcomings of being unable to clone low complexity sequence motifs such as repeats, homopolymeric nucleotide runs, and high/low GC sequences. In addition, the ability to generate libraries of high sequence variation at defined sequences is even more problematic. Methods for overcoming these limitations have been developed that are based on the synthesis and incorporation of highly pure long single stranded oligonucleotides, such as Ultramers oligonucleotides (Integrated DNA Technologies, Inc.) into double stranded clonal/non-clonal PCR products (see gBlocks® gene block fragments from Integrated DNA Technologies, Inc.). Once fully assembled, the double stranded material can be subjected to error correction methodologies to improve the fidelity of the end product.

The methods of the invention described herein provide high quality oligonucleotide subunits that are ideal for gene synthesis and improved methods to assemble said subunits into longer genetic elements. Furthermore, the genetic elements can be configured to contain regions of high variability by incorporating degenerate bases, These and other advantages of the invention, as well as additional inventive features, will be apparent from the description of the invention provided herein.

### BRIEF SUMMARY OF THE INVENTION

The methods include the synthesis of long, double stranded nucleic acid sequences containing regions of low complexity, repeating elements, sequences traditionally difficult to assemble and clone, or variable regions containing mixed bases.

In one embodiment, two or more clonal or non-clonal DNA fragments (“gBlocks” or “gene blocks”) are bound or covalently linked together with an overlapping single stranded oligonucleotide (a “bridging oligonucleotide”) optionally containing a variable region, a repeat region or a combination thereof, to form a larger DNA fragment or variable DNA fragment library. The constructed DNA fragments or libraries themselves can be joined with one or more additional DNA fragments, optionally with a bridging oligonucleotide containing further repeat or variable regions, to make longer fragments in either an iterative fashion or in a single reaction.

The bridging oligonucleotide contains overlap regions where the 3′ and the 5′ portions of the bridging oligonucleotide overlap the DNA fragments (gBlocks). Between the bridging oligonucleotide and each gBlock, the overlap can be completely or partially complementary to one strand of the gBlock, the essential element being the ability for the bridging oligonucleotide to hybridize to a strand of the gBlock and allow for strand extension. The resulting product is a larger DNA fragment comprised of a first gBlock, a double-stranded portion encoding the bridge portion of the bridging oligonucleotide, and a second gBlock (FIG. 1A). In a further embodiment, the bridging oligonucleotide contains at least one degenerate/mixed base or mismatch within the overlap region.

In a further embodiment, a second bridging oligonucleotide containing a fixed base or mixed base bridge sequence and overlap with the second gBlock and a third gBlock, can be added to incorporate more than one fixed or variable region originating from the bridge sequence into the final DNA fragment or library (FIG. 1B).

The final DNA fragments or library can then be inserted into vectors, such as bacterial DNA plasmids, and clonally amplified through methods well-known in the art.

In a further embodiment, gene blocks are synthesized or combined in such a manner as to provide 3′ and 5′ flanking sequences that enable the synthetic nucleic acid elements to be more easily inserted into a vector using an isothermal assembly method or other homologous recombination methods.

In another embodiment, a single bridging oligonucleotide can combine more than two gBlocks. The bridging oligonucleotide can be long enough to overlap an entire sufficiently complementary strand of a first gBlock, wherein the bridging oligonucleotide is longer than the first gBlock to have 3′ and 5′ ends that can serve to hybridize to a second gBlock 3′ of the first gBlock and hybridize 5′ to a third gBlock, resulting in a new fragment that encodes for at least three gBlocks as well as the bridge sequences.

In another embodiment, the component oligonucleotide(s) that are employed to synthesize the synthetic nucleic acid elements are high-fidelity (i.e., low error) oligonucleotides synthesized on supports comprised of thermoplastic polymer and controlled pore glass (CPG), wherein the amount of CPG per support by percentage is between 1-8% by weight.

### BRIEF DESCRIPTION OF THE DRAWINGS

FIG. 1A is an illustration of the use of a bridging oligonucleotide and primers to PCR assemble degenerate or low complexity sequences between two double stranded DNA fragments. FIG. 1B demonstrates how multiple bridges and double stranded DNA fragments can be used simultaneously or in a reiterative fashion to introduce more than one repeat or variable region.

FIG. 2A is an agarose gel image showing the successful generation of the full length double stranded DNA product after incorporation of the bridging oligonucleotide containing direct or indirect repeats, CAT nucleotide repeats, or homopolymeric runs of G nucleotides between two non-clonal DNA fragments (gBlocks). FIG. 2B is an agarose gel image showing the newly generated full length DNA fragments after undergoing error correction and PCR.

FIGS. 3A-3C show the ESI mass spectrum for error corrected products containing repeat regions of low complexity introduced by a bridging oligonucleotide. Both strands of the double-stranded DNA fragments were detected and the most prevalent measured mass values match the expected mass values for each strand. FIG. 3A shows the mass spectrum for construct 4 (SEQ ID 025), which contains two 64 bp direct repeats. FIG. 3B shows the mass spectrum for construct 11 (SEQ ID 032), which contains 18 CAT nucleotide direct repeats. FIG. 3C shows the mass spectrum for construct 14 (SEQ ID 035), which contains a homopolymeric run of seven G bases.

FIG. 4 shows the Sanger sequencing results of cloned products containing low complexity repeat regions before and after error correction. Correct full length clones are obtained with or without error correction, and the percentage of correct clones is increased after error correction for 7 out of 8 sequences.

FIG. 5A is an agarose gel image showing the successful assembly of a double stranded DNA fragment library after incorporation between two gBlocks of a bridging oligonucleotide containing a single NNK bridge sequence. FIGS. 5B and 5C are tables indicating the base distribution at each degenerate position obtained by next generation sequencing on an Illumina MiSeq® instrument. The results are shown as either the read count for each nucleotide at each NNK position (5B) or the percentage of times a particular base is observed at a given NNK position (5C).

FIG. 6 shows the nucleotide distribution percentages at each position for a gBlock library containing 6 tandem NNK degenerate positions obtained through next generation sequencing on an Illumina MiSeq.

FIG. 7 is an agarose gel showing the successful assembly of a gBlock library containing non-contiguous regions of degenerate bases separated by fixed DNA sequences. The correct product is marked by a star.

FIG. 8A is an illustration of the assembly of a walking library in which multiple bridging oligonucleotides, each containing a degenerate region at successive positions along the bridge sequence, are pooled and assembled with two gBlocks using PCR. FIG. 8B is an agarose gel image showing the successful assembly of a walking library before and after 10 cycles of re-amplification PCR.

FIG. 9 is an agarose gel image showing the PCR products obtained from re-amplifying for 10 or 20 cycles a double stranded gBlock library with a variable region containing 12 N mixed base positions and demonstrates the importance of limiting the number of PCR re-amplification cycles performed on a double stranded library.

### DETAILED DESCRIPTION OF THE INVENTION

Aspects of this invention relate to methods for synthesis of synthetic nucleic acid elements that may comprise genes or gene fragments. More specifically, the methods of the invention include methods of gene assembly through bridging of adjacent clonal or non-clonal double stranded DNA fragments (gBlocks) with a bridging oligonucleotide that optionally contains degenerate, variable or repeat sequences. The bridging oligonucleotide may include degenerate or mismatch bases within the overlapping regions to alter the sequence of adjacent gBlocks.

The term “oligonucleotide,” as used herein, refers to polydeoxyribonucleotides (containing 2-deoxy-D-ribose), polyribonucleotides (containing D-ribose), and to any other type of polynucleotide which is an N glycoside of a purine or pyrimidine base. There is no intended distinction in length between the terms “nucleic acid”, “oligonucleotide” and “polynucleotide”, and these terms can be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

The terms “raw material oligonucleotide” refers to the initial oligonucleotide material that is further processed, synthesized, combined, joined, modified, transformed, purified or otherwise refined to form the basis of another oligonucleotide product. The raw material oligonucleotides are typically, but not necessarily, the oligonucleotides that are directly synthesized using phosphoramidite chemistry. The term “gBlock” is a broader term to refer to double stranded DNA fragments (of clonal or non-clonal origin), sometimes referred to as gene sub-blocks or gene blocks. The synthesis of gBlocks is described in U.S. application Ser. No. 13/742,959 and is referenced herein in its entirety.

The term “base” as used herein includes purines, pyrimidines and non-natural bases and modifications well-known in the art. Purines include adenine, guanine and xanthine and modified purines such as 8-oxo-N6-methyladenine and 7-deazaxanthine. Pyrimidines include thymine, uracil and cytosine and their analogs such as 5-methylcytosine and 4,4-ethanocytosine. Non-natural bases include 5-fluorouracil, 5-bromouracil, 5-chlorouracil, 5-iodouracil, hypoxanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, nitroindole, and 2,6-diaminopurine.

The term “base” is sometimes used interchangeably with “monomer”, and in this context it refers to a single nucleic acid or oligomer unit in a nucleic acid chain.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The oligonucleotides used in the inventive methods can be synthesized using any of the methods of enzymatic or chemical synthesis known in the art, although phosphoramidite chemistry is the most common. The oligonucleotides may be synthesized on solid supports such as controlled pore glass (CPG), polystyrene beads, or membranes composed of thermoplastic polymers that may contain CPG. Oligonucleotides can also be synthesized on arrays, on a parallel microscale using microfluidics (Tian et al., Mol. BioSyst., 5, 714-722 (2009)), or known technologies that offer combinations of both (see Jacobsen et al., U.S. Pat. App. No. 2011/0172127).

Synthesis on arrays or through microfluidics offers an advantage over conventional solid support synthesis by reducing costs through lower reagent use. The scale required for gene synthesis is low, so the scale of oligonucleotide product synthesized from arrays or through microfluidics is acceptable. However, the synthesized oligonucleotides are of lesser quality than when using solid support synthesis (See Tian infra.; see also Staehler et al., U.S. Pat. App. No. 2010/0216648). High fidelity oligonucleotides are required in some embodiments of the methods of the present invention, and therefore array or microfluidic oligonucleotide synthesis will not always be compatible.

In one embodiment of the present invention, the oligonucleotides that are used for gene synthesis methods are high-fidelity oligonucleotides (average coupling efficiency is greater than 99.2%, or more preferably 99.5%). High-fidelity oligonucleotides are available commercially up to 200 bases in length (see Ultramer® oligonucleotides from Integrated DNA Technologies, Inc.). Alternatively, the oligonucleotide is synthesized using low-CPG load solid supports that provide synthesis of high-fidelity oligonucleotides while reducing reagent use. Solid support membranes are used wherein the composition of CPG in the membranes is no more than 8% of the membrane by weight. Membranes known in the art are typically 20-50% (see for example, Ngo et al., U.S. Pat. No. 7,691,316). In a further embodiment, the composition of CPG in the membranes is no more than 5% of the membrane. The membranes offer scales as low as subnanomolar scales that are ideal for the amount of oligonucleotides used as the building blocks for gene synthesis. Less reagent amounts are necessary to perform synthesis using these novel membranes. The membranes can provide as low as 100-picomole scale synthesis or less.

Other methods are known in the art to produce high-fidelity oligonucleotides. Enzymatic synthesis or the replication of existing PCR products traditionally has lower error rates than chemical synthesis of oligonucleotides due to convergent consensus within the amplifying population. However, further optimization of the phosphoramidite chemistry can achieve even greater quality oligonucleotides, which improves any gene synthesis method. A great number of advances have been achieved in the traditional four-step phosphoramidite chemistry since it was first described in the 1980's (see for example, Sierzchala, et al. J. Am. Chem. Soc., 125, 13427-13441 (2003) using peroxy anion deprotection; Hayakawa et al., U.S. Pat. No. 6,040,439 for alternative protecting groups; Azhayev et al, Tetrahedron 57, 4977-4986 (2001) for universal supports; Kozlov et al., Nucleosides, Nucleotides, and Nucleic Acids, 24 (5-7), 1037-1041 (2005) for improved synthesis of longer oligonucleotides through the use of large-pore CPG; and Damha et al., NAR, 18, 3813-3821 (1990) for improved derivitization).

Regardless of the type of synthesis, the resulting oligonucleotides may then form the smaller building blocks for longer oligonucleotides or gBlocks. As referenced earlier, the smaller oligonucleotides can be joined together using protocols known in the art, such as polymerase chain assembly (PCA), ligase chain reaction (LCR), and thermodynamically balanced inside-out synthesis (TBIO) (see Czar et al. Trends in Biotechnology, 27, 63-71 (2009)). In PCA oligonucleotides spanning the entire length of the desired longer product are annealed and extended in multiple cycles (typically about 55 cycles) to eventually achieve full-length product. LCR uses ligase enzyme to join two oligonucleotides that are both annealed to a third oligonucleotide. TBIO synthesis starts at the center of the desired product and is progressively extended in both directions by using overlapping oligonucleotides that are homologous to the forward strand at the 5′ end of the gene and against the reverse strand at the 3′ end of the gene.

Another method of synthesizing a larger double stranded DNA fragment or gBlock is to combine smaller oligonucleotides through top-strand PCR (TSP). In this method, a plurality of oligonucleotides span the entire length of a desired product and contain overlapping regions to the adjacent oligonucleotide(s). Amplification can be performed with universal forward and reverse primers, and through multiple cycles of amplification a full-length double stranded DNA product is formed. This product can then undergo optional error correction and further amplification that results in the desired double stranded DNA fragment (gBlock) end product.

In one method of TSP, the set of smaller oligonucleotides that will be combined to form the full-length desired product are between 40-200 bases long and overlap each other by at least about 15-20 bases. For practical purposes, the overlap region should be at a minimum long enough to ensure specific annealing of oligonucleotides and have a high enough melting temperature (Tₘ) to anneal at the reaction temperature employed. The overlap can extend to the point where a given oligonucleotide is completely overlapped by adjacent oligonucleotides. The amount of overlap does not seem to have any effect on the quality of the final product. The first and last oligonucleotide building block in the assembly should contain binding sites for forward and reverse amplification primers. In one embodiment, the terminal end sequence of the first and last oligonucleotide contain the same sequence of complementarity to allow for the use of universal primers.

Methods of mitigating synthesis errors are known in the art, and they optionally could be incorporated into methods of the present invention. The error correction methods include, but are not limited to, circularization methods wherein the properly assembled oligonucleotides are circularized while the other product remain linear and was enzymatically degraded (see Bang and Church, Nat. Methods, 5, 37-39 (2008)). The mismatches can be degraded using mismatch-cleaving endonucleases such as Surveyor Nuclease. Another error correction method utilizes MutS protein that binds to mismatches, thereby allowing the desired product to be separated (see Carr, P. A. et al. Nucleic Acids Res. 32, e162 (2004)).

Whether the oligonucleotides are combined through TSP or another form of assembly, the double stranded DNA gBlocks can then be combined with the bridging oligonucleotides of the present invention to produce larger DNA fragments that optionally contain one or more variable or repeat regions. The bridging oligonucleotides may contain fixed sequences to insert between gBlocks, or they may contain degenerate/mixed bases, or a combination thereof. In one embodiment the bridging oligonucleotide contains at least one mismatch within the overlap region in order to produce a large DNA fragment containing the bridge sequence and the adjacent gBlock sequences but for the substitution caused through the overlap mismatch.

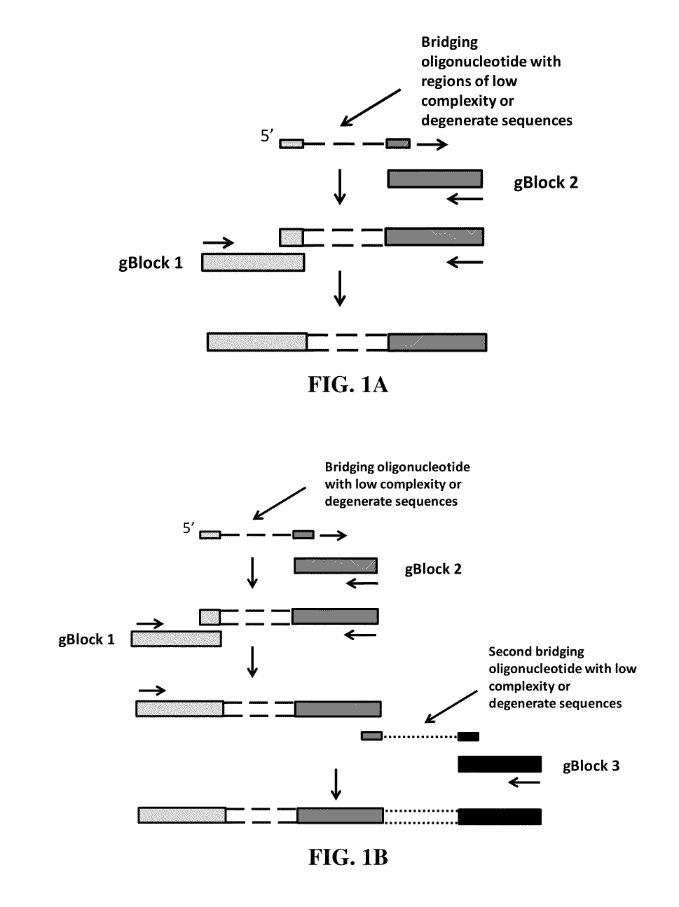

The term “bridging oligonucleotide” refers to the single stranded oligonucleotide that contains ends at least partially complementary to the adjacent gBlocks. As illustrated in FIG. 1A, the 5′-end of the bridging oligonucleotide shares complementarity with a first gBlock (a first overlap) and the 3′-end of the bridging oligonucleotide shares complementarity with a second gBlock (a second overlap). The “bridge” is the portion between the overlap regions and through PCR cycling adds additional sequence material between the adjacent gBlocks to form the final gBlock product or library. The bridge may be a fixed sequence, for example a repeat sequence, or it may contain degenerate bases. Alternatively the bridging oligonucleotide may just contain overlap with adjacent gBlocks and no internal bridge sequence, thereby combining the two gBlocks through PCR cycling without adding additional sequence between them.

In another embodiment, a single bridging oligonucleotide can combine more than two gBlocks. The bridging oligonucleotide can be long enough to overlap an entire sufficiently complementary strand of a first gBlock, wherein the bridging oligonucleotide is longer than the first gBlock to have 3′ and 5′ ends that can serve to hybridize to a second gBlock 3′ of the first gBlock and hybridize 5′ to a third gBlock, resulting in a new fragment that encodes for at least three gBlocks as well as the bridge sequences. In a further embodiment, the bridge can act as a constant variable, while the gBlock set can be diverse, such as a gBlock position using variable gBlocks for multiple promoters, or to prepare for multiple vectors.

The degenerate bases are a random mixture of multiple bases (also known as “mixed bases”), and for the purposes of this application can also refer to non-standard bases or spacers such as propanediol. For example, the degenerate bases may be an N mixture (a mixture of A, C, G and T bases), a K mixture (G and T bases), or an S mixture (G and C bases). Examples of non-standard bases include universal bases such as 3-nitropyrrole or 5-nitroindole.

The degenerate bases can be added for the purpose of increasing or reducing the GC content, or to construct a mutation library. In one embodiment a particular region of interest in a sequence is targeted to determine the effects of alternate bases on the expression of the encoded product. Only a relatively small amount of randomers inserted in the bridge could produce a large mutant library. Each N base would result in 4 different products. Each additional N base added by the bridging oligonucleotide would exponentially increase the library so that 2 N bases results in 16 combinations, 3 N bases results in 64, etc. By the time 18 N bases are inserted, the library contains over 68 billion different gene fragments. The cost of producing a library through the use of the methods of the invention is exponentially less expensive than through synthesizing each member of the library individually.

The bridging oligonucleotide will contain overlaps typically (but not limited to) 5-40 bases long on each side. The overlap is generally designed to create a bridging oligonucleotide/gBlock Tm of about 60-70° C. In one embodiment each overlap is about 15-25 bases long. Highly pure long single stranded oligonucleotides are commercially available up to 200 bases in length (e.g., Ultramer® oligonucleotides from Integrated DNA Technologies, Inc.), which would allow for 50 bases of overlap with each gBlock and up to 100 bases available for the bridge sequence. This allows for a large region (100 bases) to incorporate known sequence, degenerate bases, and combinations thereof. The degenerate bases may be consecutive, interrupted with known sequence, or concentrated in multiple areas along the bridge.

In another embodiment, degenerate or mismatch bases are incorporated into the adjacent gene block sequences through incorporating degenerate or mismatch bases within the overlap regions. In subsequent cycles of PCR to form a double-stranded product comprised of the gene block sequences and the bridge sequence, the mismatches will be incorporated into the longer product. The overlap regions can be designed to allow for adequate hybridization between the bridging oligonucleotide and the gBlock despite the mismatch.

In another embodiment, the bridging oligonucleotide is used to insert a sequence that is otherwise difficult to assemble or clone. The sequence may be difficult to assemble using PCR-based assembly methods using oligonucleotides such as TSP and is therefore added post-synthesis through the insertion of the sequence in the bridge portion of a bridging oligonucleotide.

In another embodiment, two or more bridging oligonucleotides can be combined with 3 or more gene blocks to assemble a DNA fragment or library resulting in combinations of one or more variable regions.

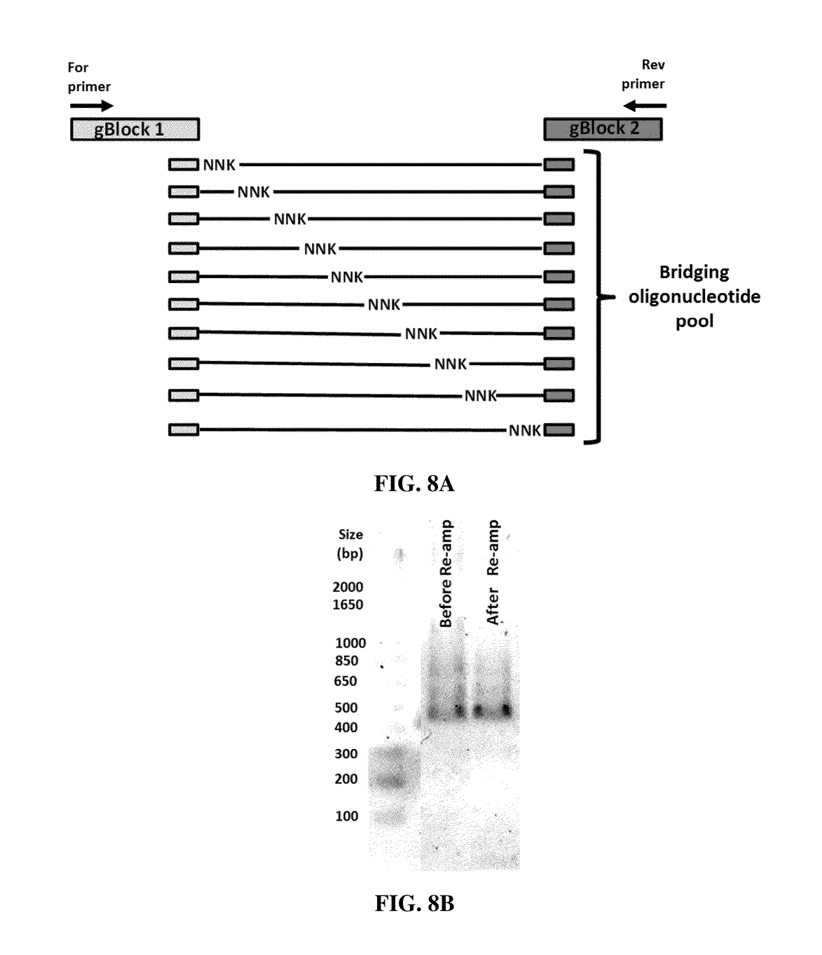

In another embodiment, a pool of individually synthesized bridging oligonucleotides can be pooled, wherein the two or more bridging oligonucleotides contain overlaps with the same two adjacent gene blocks but each contain a bridge sequence with degenerate region(s) located at successive positions along the length of the bridge sequence while keeping the rest of the bridge sequence constant (FIG. 8A). The bridging oligonucleotide pool can be utilized to assemble a library of greater depth and variation without compromising the library by use of lower quality bridging oligonucleotides that come from excessively large number of mixed base sites.

In another embodiment, a pool of individually synthesized bridging oligonucleotides can be pooled, wherein the two or more bridging oligonucleotides contain non-random variation in the bridge sequence, such as specific codon or amino acid changes.

In another embodiment, one or more bridging oligonucleotides may consist exclusively of overlap sequences with the gene blocks, thereby combining the two gene blocks through PCR cycling without adding additional sequence between the two gene blocks.

Standard PCR methods well-known in the art, following the general scheme in FIG. 1A, can be used to generate a double-stranded DNA fragment containing the bridge sequence between the adjacent gene block sequences. This end product double stranded DNA gene fragment or library can be treated as any other gene fragment described herein.

The gene blocks or libraries can then later be cloned through methods well-known in the art, such as isothermal assembly (e.g., Gibson et al. Science, 319, 1215-1220 (2008)); ligation-by-assembly or restriction cloning (e.g., Kodumal et al., Proc. Natl. Acad. Sci. U.S.A., 101, 15573-15578 (2004) and Viallalobos et al., BMC Bioinformatics, 7, 285 (2006)); TOPO TA cloning (Invitrogen/Life Tech.); blunt-end cloning; and homologous recombination (e.g., Larionov et al., Proc. Natl. Acad. Sci. U.S.A., 93, 491-496). The gene blocks can be cloned into many vectors known in the art, including but not limited to pUC57, pBluescriptII (Stratagene), pET27, Zero Blunt TOPO (Invitrogen), psiCHECK-2, pIDTSMART (Integrated DNA Technologies, Inc.), and pGEM T (Promega).

The gene blocks or libraries can be used in a variety of applications, not limited to but including protein expression (recombinant antibodies, novel fusion proteins, codon optimized short proteins, functional peptides—catalytic, regulatory, binding domains), microRNA genes, template for in vitro transcription (IVT), shRNA expression cassettes, regulatory sequence cassettes, micro-array ready cDNA, gene variants and SNPs, DNA vaccines, standards for quantitative PCR and other assays, and functional genomics (mutant libraries and unrestricted point mutations for protein mutagenesis, and deletion mutants).

One embodiment of the invention, a creation of a library in which multiple bridging oligonucleotides, each containing a degenerate region at successive positions, are pooled and assembled with double stranded DNA fragments to form a double stranded DNA walking library, could be used in a number of applications. This type of library is useful for introducing one amino acid change at a time along the sequence of interest, while keeping the other amino acids constant. This could be a useful tool in homologous recombination with gene editing technologies such as CRISPR.

The following examples further illustrate the invention but, of course, should not be construed as in any way limiting its scope.

### Example 1

This example demonstrates the incorporation of low complexity sequences into a double stranded sequence through the use of a bridging oligonucleotide and double stranded DNA fragments (gBlocks). The method is useful for constructing DNA sequences that are difficult to assemble using conventional methods due to low sequence complexity, such as large repeat regions or homopolymeric runs.

As illustrated in FIG. 1A, two double stranded non-clonal fragments, gBlock 1 and gBlock 2 (SEQ ID NO: 1 and SEQ ID NO: 2), were mixed with one single stranded DNA oligonucleotide (the bridging oligonucleotide) containing low complexity sequences. The bridge sequences contained one or more direct or indirect repeats ranging in size from 47 to 71 bases (SEQ ID NO: 3-7), 3 to 18 repeats of the CAT trimer nucleotide sequence (SEQ ID NO: 8-13) or extended stretches of homopolymeric G nucleotide (SEQ ID NO: 14-19). The 5′ end of each bridging oligonucleotide in this example contains 18 bases of overlap sequence with gBlock 1 and the 3′ end contains 18 bases of overlap with gBlock 2. Seventeen assembly reactions, each with a different bridging oligonucleotide, were setup using 25 fmoles each of gBlock 1 and gBlock 2, 250 fmoles of bridging oligonucleotide, 200 nM of each primer (SEQ ID NO: 20 and 21), 0.02 U/μl of KOD Hot-Start DNA polymerase (Novagen), 1×KOD Buffer, 1.5 mM MgSO₄, and 0.8 mM dNTPs in a final 50 μl reaction volume and subjected to PCR cycling using the following conditions: 95° C.^{3:00} (95° C.^{0:20}-61° C.^{0:10}-70° C.^{0:15})×25 cycles. The assembly PCR resulted in 17 constructs (SEQ ID NO: 22-38) with the bridging oligonucleotide sequence incorporated between gBlock 1 and gBlock 2.

TABLE I
SEQ ID listing of oligonucleotides used in Examples

| | |
|---|---|
| gBlock 1 | AATGATACGGCGACCACCGAGATCTACACTCTTTCCCTACACGACGCTCTT |
| (SEQ ID 001) | CCGATCTGCTAGCGCCGGATCTTCGTGACAAGACCATCACCACTTGACAGT |
| | TGGCCGTCGACCCTGCACCTGGTCCTGCGTCTGAGAGGTGGT |
| gBlock 2 | TCGTATGAATTCGCGGCCGCTTCTAGAGCCACAATTCAGCAAATTGTGAAC |
| (SEQ ID 002) | ATCATCTCCCTGGTTGCTCCTGTCAGTAAGTAATGAGATCGGAAGAGCACA |
| | CGTCTGAACTCCAGTCACCAGATCATCTCGTATGCCGTCTTCTGCTTG |
| Bridge 1-71 base repeat | CTGCGTCTGAGAGGTGGTACATGGGTGAACTTACTTGCATACCAAGTTGA |
| (SEQ ID 003) | TACTTGAATAACCATCTGAAAGTGGTACTTGATCATTTTACATGGGTGAAC |
| | TTACTTGCATACCAAGTTGATACTTGAATAACCATCTGAAAGTGGTACTTG |
| | ATCATTTTTCGTATGAATTCGCGGCC |
| Bridge 2-47 base repeat | CTGCGTCTGAGAGGTGGTCATCACCATCACCATCACCATCACCACCATCAT |
| (SEQ ID 004) | TAGATGAATATGAAACATTTTCACTTGTTCTTCCTACTCACGCTTCTGTTTCT |
| | TACACCCAGGATTCAGGCACATCATCACCATCACCATCACCATCACCACCA |
| | TCATTAGATGAATATGAATCGTATGAATTCGCGGCC |
| Bridge 3-50 base repeat | CTGCGTCTGAGAGGTGGTCAAGGCATAAAACCAAATCTCATTCTCTTTCTT |
| (SEQ ID 005) | CTCTATTCTTTGCAGCCATGGGTAATTACCAACAACAACAAACAACAAACA |
| | ACATTACAATTAATAAAACCAAATCTCATTCTCTTTCTTCTCTATTCTTTGCA |
| | GCCATGGGTCTGCAGTCGTATGAATTCGCGGCC |
| Bridge 4-64 base repeat | CTGCGTCTGAGAGGTGGTTATTGCATACCCGTTTTTAATAAAATACATTGC |
| (SEQ ID 006) | ATACCCTCTTTTAATAAAAAATATTGCATACTTTGACGAAATATTGCATACC |
| | CGTTTTTAATAAAATACATTGCATACCCTCTTTTAATAAAAAATATTGCATA |
| | CTCGTATGAATTCGCGGCC |
| Bridge 5-65 base repeat | CTGCGTCTGAGAGGTGGTACGAACCAGAGGATCCCTGCTAGCCAATGGG |
| (SEQ ID 007) | GCGATCGCCCACAATTGCGGTGGCGGAAAATTTAAAGGATCTGGAGGGG |
| | GCATCATCAGGATCCCTGCTAGCCAATGGGGCGATCGCCCACAATTGCGG |
| | TGGCGGAAAATTTAAAGGATCTGGTGGGGGAGGTTCGTATGAATTCGCG |
| | GCC |
| Bridge 6-3 CAT repeats | CTGCGTCTGAGAGGTGGTTCATCCGCGAGACCACACGCCATCATCATCAC |
| (SEQ ID 008) | GTGAAGATGATATCGTTTCGTATGAATTCGCGGCC |
| Bridge 7-6 CAT repeats | CTGCGTCTGAGAGGTGGTTCATCCGCGAGACCACACGCCATCATCATCATC |
| (SEQ ID 009) | ATCATCACGTGAAGATGATATCGTTTCGTATGAATTCGCGGCC |
| Bridge 8-9 CAT repeats | CTGCGTCTGAGAGGTGGTTCATCCGCGAGACCACACGCCATCATCATCATC |
| (SEQ ID 010) | ATCATCATCATCATCACGTGAAGATGATATCGTTTCGTATGAATTCGCGGCC |
| Bridge 9-12 CAT repeats | CTGCGTCTGAGAGGTGGTTCATCCGCGAGACCACACGCCATCATCATCATC |
| (SEQ ID 011) | ATCATCATCATCATCATCATCATCACGTGAAGATGATATCGTTTCGTATGAA |
| | TTCGCGGCC |
| Bridge 10-15 CAT repeats | CTGCGTCTGAGAGGTGGTTCATCCGCGAGACCACACGCCATCATCATCATC |
| (SEQ ID 012) | ATCATCATCATCATCATCATCATCATCATCATCACGTGAAGATGATATCGTT |
| | TCGTATGAATTCGCGGCC |
| Bridge 11-18 CAT repeats | CTGCGTCTGAGAGGTGGTTCATCCGCGAGACCACACGCCATCATCATCATC |
| (SEQ ID 013) | ATCATCATCATCATCATCATCATCATCATCATCATCATCATCACGTGAAGAT |
| | GATATCGTTTCGTATGAATTCGCGGCC |
| Bridge 12-5G | CTGCGTCTGAGAGGTGGTTCATCCGCGAGACCACACGCGGGGGCACGTG |
| (SEQ ID 014) | AAGATGATATCGTTTCGTATGAATTCGCGGCC |
| Bridge 13-6G | CTGCGTCTGAGAGGTGGTTCATCCGCGAGACCACACGCGGGGGGCACGT |
| (SEQ ID 015) | GAAGATGATATCGTTTCGTATGAATTCGCGGCC |
| Bridge 14-7G | CTGCGTCTGAGAGGTGGTTCATCCGCGAGACCACACGCGGGGGGGCACG |
| (SEQ ID 016) | TGAAGATGATATCGTTTCGTATGAATTCGCGGCC |
| Bridge 15-8G | CTGCGTCTGAGAGGTGGTTCATCCGCGAGACCACACGCGGGGGGGGCAC |
| (SEQ ID 017) | GTGAAGATGATATCGTTTCGTATGAATTCGCGGCC |
| Bridge 16-9G | CTGCGTCTGAGAGGTGGTTCATCCGCGAGACCACACGCGGGGGGGGGCA |
| (SEQ ID 018) | CGTGAAGATGATATCGTTTCGTATGAATTCGCGGCC |
| Bridge 17-10G | CTGCGTCTGAGAGGTGGTTCATCCGCGAGACCACACGCGGGGGGGGGGC |
| (SEQ ID 019) | ACGTGAAGATGATATCGTTTCGTATGAATTCGCGGCC |
| For primer | AATGATACGGCGACCACCG |
| (SEQ ID 020) | |
| Rev primer | CAAGCAGAAGACGGCATACGA |
| (SEQ ID 021) | |
| Construct 1-436 bp | AATGATACGGCGACCACCGAGATCTACACTCTTTCCCTACACGACGCTCTT |
| (SEQ ID 022) | CCGATCTGCTAGCGCCGGATCTTCGTGACAAGACCATCACCACTTGACAGT |
| | TGGCCGTCGACCCTGCACCTGGTCCTGCGTCTGAGAGGTGGTACATGGGT |
| | GAACTTACTTGCATACCAAGTTGATACTTGAATAACCATCTGAAAGTGGTA |
| | CTTGATCATTTTACATGGGTGAACTTACTTGCATACCAAGTTGATACTTGAA |
| | TAACCATCTGAAAGTGGTACTTGATCATTTTTCGTATGAATTCGCGGCCGC |
| | TTCTAGAGCCACAATTCAGCAAATTGTGAACATCATCTCCCTGGTTGCTCCT |
| | GTCAGTAAGTAATGAGATCGGAAGAGCACACGTCTGAACTCCAGTCACCG |
| | ATGTATCTCGTATGCCGTCTTCTGCTTG |
| Construct 2-449 bp | AATGATACGGCGACCACCGAGATCTACACTCTTTCCCTACACGACGCTCTT |
| (SEQ ID 023) | CCGATCTGCTAGCGCCGGATCTTCGTGACAAGACCATCACCACTTGACAGT |
| | TGGCCGTCGACCCTGCACCTGGTCCTGCGTCTGAGAGGTGGTCATCACCAT |
| | CACCATCACCATCACCACCATCATTAGATGAATATGAAACATTTTCACTTGT |
| | TCTTCCTACTCACGCTTCTGTTTCTTACACCCAGGATTCAGGCACATCATCA |
| | CCATCACCATCACCATCACCACCATCATTAGATGAATATGAATCGTATGAA |
| | TTCGCGGCCGCTTCTAGAGCCACAATTCAGCAAATTGTGAACATCATCTCC |
| | CTGGTTGCTCCTGTCAGTAAGTAATGAGATCGGAAGAGCACACGTCTGAA |
| | CTCCAGTCACCGATGTATCTCGTATGCCGTCTTCTGCTTG |
| Construct 3-446 bp | AATGATACGGCGACCACCGAGATCTACACTCTTTCCCTACACGACGCTCTT |
| (SEQ ID 024) | CCGATCTGCTAGCGCCGGATCTTCGTGACAAGACCATCACCACTTGACAGT |
| | TGGCCGTCGACCCTGCACCTGGTCCTGCGTCTGAGAGGTGGTCAAGGCAT |
| | AAAACCAAATCTCATTCTCTTTCTTCTCTATTCTTTGCAGCCATGGGTAATTA |
| | CCAACAACAACAAACAACAAACAACATTACAATTAATAAAACCAAATCTCA |
| | TTCTCTTTCTTCTCTATTCTTTGCAGCCATGGGTCTGCAGTCGTATGAATTC |
| | GCGGCCGCTTCTAGAGCCACAATTCAGCAAATTGTGAACATCATCTCCCTG |
| | GTTGCTCCTGTCAGTAAGTAATGAGATCGGAAGAGCACACGTCTGAACTC |
| | CAGTCACCGATGTATCTCGTATGCCGTCTTCTGCTTG |
| Construct 4-432 bp | AATGATACGGCGACCACCGAGATCTACACTCTTTCCCTACACGACGCTCTT |
| (SEQ ID 025) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTATTGCATA |
| | CCCGTTTTTAATAAAATACATTGCATACCCTCTTTTAATAAAAAATATTGCA |
| | TACTTTGACGAAATATTGCATACCCGTTTTTAATAAAATACATTGCATACCC |
| | TCTTTTAATAAAAAATATTGCATACTCGTATGAATTCGCGGCCGCTTCTAGA |
| | GCCACAATTCAGCAAATTGTGAACATCATCTCCCTGGTTGCTCCTGTCAGT |
| | AAGTAATGAGATCGGAAGAGCACACGTCTGAACTCCAGTCACCGATGTAT |
| | CTCGTATGCCGTCTTCTGCTTG |
| Construct 5-458 bp | AATGATACGGCGACCACCGAGATCTACACTCTTTCCCTACACGACGCTCTT |
| (SEQ ID 026) | CCGATCTGCTAGCGCCGGATCTTCGTGACAAGACCATCACCACTTGACAGT |
| | TGGCCGTCGACCCTGCACCTGGTCCTGCGTCTGAGAGGTGGTACGAACCA |
| | GAGGATCCCTGCTAGCCAATGGGGCGATCGCCCACAATTGCGGTGGCGG |
| | AAAATTTAAAGGATCTGGAGGGGGCATCATCAGGATCCCTGCTAGCCAAT |
| | GGGGCGATCGCCCACAATTGCGGTGGCGGAAAATTTAAAGGATCTGGTG |
| | GGGGAGGTTCGTATGAATTCGCGGCCGCTTCTAGAGCCACAATTCAGCAA |
| | ATTGTGAACATCATCTCCCTGGTTGCTCCTGTCAGTAAGTAATGAGATCGG |
| | AAGAGCACACGTCTGAACTCCAGTCACCGATGTATCTCGTATGCCGTCTTC |
| | TGCTTG |
| Construct 6-343 bp | AATGATACGGCGACCACCGAGATCTACACTCTTTCCCTACACGACGCTCTT |
| (SEQ ID 027) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCCATCATCATCACGTGAAGATGATATCGTTTCGTATGAAT |
| | TCGCGGCCGCTTCTAGAGCCACAATTCAGCAAATTGTGAACATCATCTCCC |
| | TGGTTGCTCCTGTCAGTAAGTAATGAGATCGGAAGAGCACACGTCTGAAC |
| | TCCAGTCACCGATGTATCTCGTATGCCGTCTTCTGCTTG |
| Construct 7-352 bp | AATGATACGGCGACCACCGAGATCTACACTCTTTCCCTACACGACGCTCTT |
| (SEQ ID 028) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCCATCATCATCATCATCATCACGTGAAGATGATATCGTTT |
| | CGTATGAATTCGCGGCCGCTTCTAGAGCCACAATTCAGCAAATTGTGAACA |
| | TCATCTCCCTGGTTGCTCCTGTCAGTAAGTAATGAGATCGGAAGAGCACAC |
| | GTCTGAACTCCAGTCACCGATGTATCTCGTATGCCGTCTTCTGCTTG |
| Construct 8-361 bp | AATGATACGGCGACCACCGAGATCTACACTCTTTCCCTACACGACGCTCTT |
| (SEQ ID 029) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCCATCATCATCATCATCATCATCATCATCACGTGAAGATG |
| | ATATCGTTTCGTATGAATTCGCGGCCGCTTCTAGAGCCACAATTCAGCAAA |
| | TTGTGAACATCATCTCCCTGGTTGCTCCTGTCAGTAAGTAATGAGATCGGA |
| | AGAGCACACGTCTGAACTCCAGTCACCGATGTATCTCGTATGCCGTCTTCT |
| | GCTTG |
| Construct 9-370 bp | AATGATACGGCGACCACCGAGATCTACACTCTTTCCCTACACGACGCTCTT |
| (SEQ ID 030) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCCATCATCATCATCATCATCATCATCATCATCATCATCACG |
| | TGAAGATGATATCGTTTCGTATGAATTCGCGGCCGCTTCTAGAGCCACAAT |
| | TCAGCAAATTGTGAACATCATCTCCCTGGTTGCTCCTGTCAGTAAGTAATG |
| | AGATCGGAAGAGCACACGTCTGAACTCCAGTCACCGATGTATCTCGTATG |
| | CCGTCTTCTGCTTG |
| Construct 10-379 bp | AATGATACGGCGACCACCGAGATCTACACTCTTTCCCTACACGACGCTCTT |
| (SEQ ID 031) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCCATCATCATCATCATCATCATCATCATCATCATCATCATC |
| | ATCATCACGTGAAGATGATATCGTTTCGTATGAATTCGCGGCCGCTTCTAG |
| | AGCCACAATTCAGCAAATTGTGAACATCATCTCCCTGGTTGCTCCTGTCAG |
| | TAAGTAATGAGATCGGAAGAGCACACGTCTGAACTCCAGTCACCGATGTA |
| | TCTCGTATGCCGTCTTCTGCTTG |
| Construct 11-388 bp | AATGATACGGCGACCACCGAGATCTACACTCTTTCCCTACACGACGCTCTT |
| (SEQ ID 032) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCCATCATCATCATCATCATCATCATCATCATCATCATCATC |
| | ATCATCATCATCATCACGTGAAGATGATATCGTTTCGTATGAATTCGCGGC |
| | CGCTTCTAGAGCCACAATTCAGCAAATTGTGAACATCATCTCCCTGGTTGC |
| | TCCTGTCAGTAAGTAATGAGATCGGAAGAGCACACGTCTGAACTCCAGTC |
| | ACCGATGTATCTCGTATGCCGTCTTCTGCTTG |
| Construct 12-339 bp | AATGATACGGCGACCACCGAGATCTACACTCTTTCCCTACACGACGCTCTT |
| (SEQ ID 033) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCGGGGGCACGTGAAGATGATATCGTTTCGTATGAATTCG |
| | CGGCCGCTTCTAGAGCCACAATTCAGCAAATTGTGAACATCATCTCCCTGG |
| | TTGCTCCTGTCAGTAAGTAATGAGATCGGAAGAGCACACGTCTGAACTCC |
| | AGTCACCGATGTATCTCGTATGCCGTCTTCTGCTTG |
| Construct 13-340 bp | AATGATACGGCGACCACCGAGATCTACACTCTTTCCCTACACGACGCTCTT |
| (SEQ ID 034) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCGGGGGGCACGTGAAGATGATATCGTTTCGTATGAATTC |
| | GCGGCCGCTTCTAGAGCCACAATTCAGCAAATTGTGAACATCATCTCCCTG |
| | GTTGCTCCTGTCAGTAAGTAATGAGATCGGAAGAGCACACGTCTGAACTC |
| | CAGTCACCGATGTATCTCGTATGCCGTCTTCTGCTTG |
| Construct 14-341 bp | AATGATACGGCGACCACCGAGATCTACACTCTTTCCCTACACGACGCTCTT |
| (SEQ ID 035) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCGGGGGGGCACGTGAAGATGATATCGTTTCGTATGAATT |
| | CGCGGCCGCTTCTAGAGCCACAATTCAGCAAATTGTGAACATCATCTCCCT |
| | GGTTGCTCCTGTCAGTAAGTAATGAGATCGGAAGAGCACACGTCTGAACT |
| | CCAGTCACCGATGTATCTCGTATGCCGTCTTCTGCTTG |
| Construct 15-342 bp | AATGATACGGCGACCACCGAGATCTACACTCTTTCCCTACACGACGCTCTT |
| (SEQ ID 036) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCGGGGGGGGCACGTGAAGATGATATCGTTTCGTATGAA |
| | TTCGCGGCCGCTTCTAGAGCCACAATTCAGCAAATTGTGAACATCATCTCC |
| | CTGGTTGCTCCTGTCAGTAAGTAATGAGATCGGAAGAGCACACGTCTGAA |
| | CTCCAGTCACCGATGTATCTCGTATGCCGTCTTCTGCTTG |
| Construct 16-343 bp | AATGATACGGCGACCACCGAGATCTACACTCTTTCCCTACACGACGCTCTT |
| (SEQ ID 037) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCGGGGGGGGGCACGTGAAGATGATATCGTTTCGTATGA |
| | ATTCGCGGCCGCTTCTAGAGCCACAATTCAGCAAATTGTGAACATCATCTC |
| | CCTGGTTGCTCCTGTCAGTAAGTAATGAGATCGGAAGAGCACACGTCTGA |
| | ACTCCAGTCACCGATGTATCTCGTATGCCGTCTTCTGCTTG |
| Construct 17-344 bp | AATGATACGGCGACCACCGAGATCTACACTCTTTCCCTACACGACGCTCTT |
| (SEQ ID 038) | CCGATCTGCTAGCGCCGGATCTTCGTGACAAGACCATCACCACTTGACAGT |
| | TGGCCGTCGACCCTGCACCTGGTCCTGCGTCTGAGAGGTGGTTCATCCGC |
| | GAGACCACACGCGGGGGGGGGGCACGTGAAGATGATATCGTTTCGTATG |
| | AATTCGCGGCCGCTTCTAGAGCCACAATTCAGCAAATTGTGAACATCATCT |
| | CCCTGGTTGCTCCTGTCAGTAAGTAATGAGATCGGAAGAGCACACGTCTG |
| | AACTCCAGTCACCGATGTATCTCGTATGCCGTCTTCTGCTTG |
| P5 gBlock 1 | AATGATACGGCGACCACCGAGATCTACACTCTTTCCCTACACGACGCTCTT |
| (SEQ ID 039) | CCGATCTTACGACTCACTATAGGGAGACCCAAGCTGGCTAGCGCCGGATC |
| | TTCGTGACAAGACCATCACCACTTGACAGTTGGCCGTCGACCCTGCACCTG |
| | GTCCTGCGTCTGAGAGGTGGT |
| P7AD002 gBlock 2 | TCGTATGAATTCGCGGCCGCTTCTAGAGCCACAATTCAGCAAATTGTGAAC |
| (SEQ ID 040) | ATCATCTCCCTGGTTGCTCCTGTCAGTAAGTAATGAATACTAGTAGCGGCC |
| | GCTGCAGGCTAACAGATCGGAAGAGCACACGTCTGAACTCCAGTCACCGA |
| | TGTATCTCGTATGCCGTCTTCTGCTTG |
| 1NNK Bridge | CTGCGTCTGAGAGGTGGTNNKTCGTATGAATTCGCGGCC |
| (SEQ ID 041) | |
| P5 For primer | AATGATACGGCGACCACCG |
| (SEQ ID 042) | |
| P7 Rev primer | CAAGCAGAAGACGGCATACGA |
| (SEQ ID 043) | |
| 1NNK gBlock library | AATGATACGGCGACCACCGAGATCTACACTCTTTCCCTACACGACGCTCTT |
| (SEQ ID 044) | CCGATCTTACGACTCACTATAGGGAGACCCAAGCTGGCTAGCGCCGGATC |
| | TTCGTGACAAGACCATCACCACTTGACAGTTGGCCGTCGACCCTGCACCTG |
| | GTCCTGCGTCTGAGAGGTGGTNNKTCGTATGAATTCGCGGCCGCTTCTAG |
| | AGCCACAATTCAGCAAATTGTGAACATCATCTCCCTGGTTGCTCCTGTCAG |
| | TAAGTAATGAATACTAGTAGCGGCCGCTGCAGGCTAACAGATCGGAAGA |
| | GCACACGTCTGAACTCCAGTCACCGATGTATCTCGTATGCCGTCTTCTGCTTG |
| P7AD009 gBlock 2 | TCGTATGAATTCGCGGCCGCTTCTAGAGCCACAATTCAGCAAATTGTGAAC |
| (SEQ ID 045) | ATCATCTCCCTGGTTGCTCCTGTCAGTAAGTAATGAATACTAGTAGCGGCC |
| | GCTGCAGGCTAACAGATCGGAAGAGCACACGTCTGAACTCCAGTCACGAT |
| | CAGATCTCGTATGCCGTCTTCTGCTTG |
| 6NNK Bridge | CTGCGTCTGAGAGGTGGTNNKNNKNNKNNKNNKNNKTCGTATGAATTC |
| (SEQ ID 046) | GCGGCC |
| 6NNK gBlock library | AATGATACGGCGACCACCGAGATCTACACTCTTTCCCTACACGACGCTCTT |
| (SEQ ID 047) | CCGATCTTACGACTCACTATAGGGAGACCCAAGCTGGCTAGCGCCGGATC |
| | TTCGTGACAAGACCATCACCACTTGACAGTTGGCCGTCGACCCTGCACCTG |
| | GTCCTGCGTCTGAGAGGTGGTNNKNNKNNKNNKNNKNNKTCGTATGAA |
| | TTCGCGGCCGCTTCTAGAGCCACAATTCAGCAAATTGTGAACATCATCTCC |
| | CTGGTTGCTCCTGTCAGTAAGTAATGAATACTAGTAGCGGCCGCTGCAGG |
| | CTAACAGATCGGAAGAGCACACGTCTGAACTCCAGTCACGATCAGATCTC |
| | GTATGCCGTCTTCTGCTTG |
| GFP-A gBlock 1 | TGCTGCTCCTCGCTGCCCAGCCGGCGATGGCCATGGTGAGCAAGGGCGA |
| (SEQ ID 048) | GGAGCTGTTCACCGGGGTGGTGCCCATCCTGGTCGAGCTGGACGGCGAC |
| | GTAAACGGCCACAAGTTCAGCGTGTCCGGCGAGGGCGAGGGCGATGCCA |
| | CCTACGGCAAGCTGACCCTGAAGTTCATCTGCACCACCGGCAAGCTGCCC |
| | GTGCCCTGGCCCACCCTCGTGACCACC |
| GFP-A gBlock 2 | CGCTACCCCGACCACATGAAGCAGCACGACTTCTTCAAGTCCGCCATGCCC |
| (SEQ ID 049) | GAAGGCTACGTCCAGGAGCGCACCATCTTCTTCAAGGACGACGGCAACTA |
| | CAAGACCCGCGCCGAGGTGAAGTTCGAGGGCGACACCCTGGTGAACCGC |
| | ATCGAGCTGAAGGGCATCGACTTCAAGGAGGACGGCAACATCC |
| GFP-A Bridge | CCCACCCTCGTGACCACCNNKNNKTACGGCNNKCAGTGCTTCNNKCGCTA |
| (SEQ ID 050) | CCCCGACCACATG |
| GFP-A For primer | TGCTGCTCCTCGCTGC |
| (SEQ ID 051) | |
| GFP-A Rev primer | GGATGTTGCCGTCCTCCTTG |
| (SEQ ID 052) | |
| GFP-A 444 bp library | TGCTGCTCCTCGCTGCCCAGCCGGCGATGGCCATGGTGAGCAAGGGCGA |
| (SEQ ID 053) | GGAGCTGTTCACCGGGGTGGTGCCCATCCTGGTCGAGCTGGACGGCGAC |
| | GTAAACGGCCACAAGTTCAGCGTGTCCGGCGAGGGCGAGGGCGATGCCA |
| | CCTACGGCAAGCTGACCCTGAAGTTCATCTGCACCACCGGCAAGCTGCCC |
| | GTGCCCTGGCCCACCCTCGTGACCACCNNKNNKTACGGCNNKCAGTGCTT |
| | CNNKCGCTACCCCGACCACATGAAGCAGCACGACTTCTTCAAGTCCGCCAT |
| | GCCCGAAGGCTACGTCCAGGAGCGCACCATCTTCTTCAAGGACGACGGCA |
| | ACTACAAGACCCGCGCCGAGGTGAAGTTCGAGGGCGACACCCTGGTGAA |
| | CCGCATCGAGCTGAAGGGCATCGACTTCAAGGAGGACGGCAACATCC |
| V8 gBlock 1 | GCGGAGGGTCGGCTAGCGGTCAAGTTCAGTTGGTTCAATCAGGTGCGGA |
| (SEQ ID 054) | AGTTAAAAAGCCTGGTGCTTCTGTTAAGGTTTCTTGTAAAGCCTCTGGCTA |
| | TACTTTTACGGGTTATTACATGCATTGGGTAAGACAGGCTCCCGGTCAGG |
| | GTTTGGAATGGATGGGTTGGATTAACCCAAACTCTGGTGGAACTAACTAT |
| | GCTCAAAAATTCCAAGGTAGAGTTAC |
| V8 gBlock 2 | TTGTCACGTTTGAGGTCTGATGATACTGCTGTTTATTACTGTGCTAGAGGT |
| (SEQ ID 055) | AAGAACTCTGATTACAATTGGGATTTCCAACATTGGGGCCAGGGCACTTT |
| | GGTTACTGTTTCAAGTGGTGGTGGAGGATCCGGCGGTGGTGTCGTACGG |
| V8 Bridge 1 | GCTCAAAAATTCCAAGGTAGAGTTACCATGNNKAGGGATACTTCTATATCT |
| (SEQ ID 056) | ACTGCTTATATGGAATTGTCACGTTTGAGGTCTGATG |
| V8 Bridge 2 | GCTCAAAAATTCCAAGGTAGAGTTACTATGACANNKGACACTTCTATATCT |
| (SEQ ID 057) | ACTGCTTATATGGAATTGTCACGTTTGAGGTCTGATG |
| V8 Bridge 3 | GCTCAAAAATTCCAAGGTAGAGTTACTATGACTAGGNNKACATCTATATCT |
| (SEQ ID 058) | ACTGCTTATATGGAATTGTCACGTTTGAGGTCTGATG |
| V8 Bridge 4 | GCTCAAAAATTCCAAGGTAGAGTTACTATGACTAGAGACNNKTCAATATC |
| (SEQ ID 059) | TACTGCTTATATGGAATTGTCACGTTTGAGGTCTGATG |
| V8 Bridge 5 | GCTCAAAAATTCCAAGGTAGAGTTACTATGACTAGAGATACANNKATTTCT |
| (SEQ ID 060) | ACTGCTTATATGGAATTGTCACGTTTGAGGTCTGATG |
| V8 Bridge 6 | GCTCAAAAATTCCAAGGTAGAGTTACTATGACTAGAGATACTTCANNKTC |
| (SEQ ID 061) | AACTGCTTATATGGAATTGTCACGTTTGAGGTCTGATG |
| V8 Bridge 7 | GCTCAAAAATTCCAAGGTAGAGTTACTATGACTAGAGATACTTCTATTNNK |
| (SEQ ID 062) | ACAGCTTATATGGAATTGTCACGTTTGAGGTCTGATG |
| V8 Bridge 8 | GCTCAAAAATTCCAAGGTAGAGTTACTATGACTAGAGATACTTCTATATCA |
| (SEQ ID 063) | NNKGCATATATGGAATTGTCACGTTTGAGGTCTGATG |
| V8 Bridge 9 | GCTCAAAAATTCCAAGGTAGAGTTACTATGACTAGAGATACTTCTATATCT |
| (SEQ ID 064) | ACANNKTACATGGAATTGTCACGTTTGAGGTCTGATG |
| V8 Bridge 10 | GCTCAAAAATTCCAAGGTAGAGTTACTATGACTAGAGATACTTCTATATCT |
| (SEQ ID 065) | ACTGCANNKATGGAGTTGTCACGTTTGAGGTCTGATG |
| V8 For primer | GCGGAGGGTCGGCTAG |
| (SEQ ID 066) | |
| V8 Rev primer | CACCACCGCCGGATCC |
| (SEQ ID 067) | |
| AD For primer | GCCTTGCCAGCCCGCTC |
| (SEQ ID 068) | |
| AD Rev primer | GCCTCCCTCGCGCCATC |
| (SEQ ID 069) | |
| AD7 gBlock 1 | GCCTTGCCAGCCCGCTCAGGCATAACTTGGACATGCCAACTTGGAAGGGA |
| (SEQ ID 070) | GAACGAAGTCAGTCATCAGGCAGACTGGGTCATCTGCTGAAATCACTTGT |
| | GATCTTGCTGAAGGAAGTAACGGCTACATCCACTGGTACCTACACCAGGA |
| | GGGGAAGGCCCCACAGCGTCTTCAGTACTATGACTCCTACAACTCCAAGG |
| | TTGTGTTGGAATCAGGAGTCAGTCCAGGGAAGTATTATACTTACGCAAGC |
| | ACAAGGAACAACTTGAGATTGATACTGCGAAATCTAATTGAAAATGACTTT |
| | GGGGTCTATTACTGTGCCACCTGGGTCGAC |
| AD7 gBlock 2 | GCATAACTTGGACATGAGTGATTGGATCAAGACGTTTGCAAAAGGGACTA |
| (SEQ ID 071) | GGCTCATAGTAACTTCGCCTGGTAAGTAATTTTTTTTCTGTTTTTATTCCAGT |
| | AATGAAAAACTGAGCATAACTTGGACATGCTGATGGCGCGAGGGAGGC |
| AD7 Bridge | CTGTGCCACCTGGGTCGACNNNNNNNNNNNNGCATAACTTGGACATGA |
| (SEQ ID 072) | GTGATTGG |
| AD7 Library | GCCTTGCCAGCCCGCTCAGGCATAACTTGGACATGCCAACTTGGAAGGGA |
| (SEQ ID 073) | GAACGAAGTCAGTCATCAGGCAGACTGGGTCATCTGCTGAAATCACTTGT |
| | GATCTTGCTGAAGGAAGTAACGGCTACATCCACTGGTACCTACACCAGGA |
| | GGGGAAGGCCCCACAGCGTCTTCAGTACTATGACTCCTACAACTCCAAGG |
| | TTGTGTTGGAATCAGGAGTCAGTCCAGGGAAGTATTATACTTACGCAAGC |
| | ACAAGGAACAACTTGAGATTGATACTGCGAAATCTAATTGAAAATGACTTT |
| | GGGGTCTATTACTGTGCCACCTGGGTCGACNNNNNNNNNNNNGCATAA |
| | CTTGGACATGAGTGATTGGATCAAGACGTTTGCAAAAGGGACTAGGCTCA |
| | TAGTAACTTCGCCTGGTAAGTAATTTTTTTTCTGTTTTTATTCCAGTAATGA |
| | AAAACTGAGCATAACTTGGACATGCTGATGGCGCGAGGGAGGC |
| AD8 gBlock 1 | GCCTTGCCAGCCCGCTCAGACGTACTCTGGACATGTAGAGCAACCTCAAAT |
| (SEQ ID 074) | TTCCAGTACTAAAACGCTGTCAAAAACAGCCCGCCTGGAATGTGTGGTGT |
| | CTGGAATAACAATTTCTGCAACATCTGTATATTGGTATCGAGAGAGACCTG |
| | GTGAAGTCATACAGTTCCTGGTGTCCATTTCATATGACGGCACTGTCAGAA |
| | AGGAATCCGGCATTCCGTCAGGCAAATTTGAGGTGGATAGGATACCTGAA |
| | ACGTCTACATCCACTCTCACCATTCACAATGTAGAGAAACAGGACATAGCT |
| | ACCTACTACTGTGCCTTGTGGGTCGAC |
| AD8 gBlock 2 | ACGTACTCTGGACATGAGTGATTGGATCAAGACGTTTGCAAAAGGGACTA |
| (SEQ ID 075) | GGCTCATAGTAACTTCGCCTGGTAAGTAATTTTTTTTCTGTTTTTATTCCAGT |
| | AATGAAAAACTGAACGTACTCTGGACATGCTGATGGCGCGAGGGAGGC |
| AD8 Bridge | CTGTGCCTTGTGGGTCGACNNNNNNNNNNNNACGTACTCTGGACATGA |
| (SEQ ID 076) | GTG |
| AD8 Library | GCCTTGCCAGCCCGCTCAGACGTACTCTGGACATGTAGAGCAACCTCAAAT |
| (SEQ ID 077) | TTCCAGTACTAAAACGCTGTCAAAAACAGCCCGCCTGGAATGTGTGGTGT |
| | CTGGAATAACAATTTCTGCAACATCTGTATATTGGTATCGAGAGAGACCTG |
| | GTGAAGTCATACAGTTCCTGGTGTCCATTTCATATGACGGCACTGTCAGAA |
| | AGGAATCCGGCATTCCGTCAGGCAAATTTGAGGTGGATAGGATACCTGAA |
| | ACGTCTACATCCACTCTCACCATTCACAATGTAGAGAAACAGGACATAGCT |
| | ACCTACTACTGTGCCTTGTGGGTCGACNNNNNNNNNNNNACGTACTCTG |
| | GACATGAGTGATTGGATCAAGACGTTTGCAAAAGGGACTAGGCTCATAGT |
| | AACTTCGCCTGGTAAGTAATTTTTTTTCTGTTTTTATTCCAGTAATGAAAAA |
| | CTGAACGTACTCTGGACATGCTGATGGCGCGAGGGAGGC |
| AD9 gBlock 1 | GCCTTGCCAGCCCGCTCAGCTTCTAAGTGGACATGTGGAGCAGTTCCAGCT |
| (SEQ ID 078) | ATCCATTTCCACGGAAGTCAAGAAAAGTATTGACATACCTTGCAAGATATC |
| | GAGCACAAGGTTTGAAACAGATGTCATTCACTGGTACCGGCAGAAACCAA |
| | ATCAGGCTTTGGAGCACCTGATCTATATTGTCTCAACAAAATCCGCAGCTC |
| | GACGCAGCATGGGTAAGACAAGCAACAAAGTGGAGGCAAGAAAGAATTC |
| | TCAAACTCTCACTTCAATCCTTACCATCAAGTCCGTAGAGAAAGAAGACAT |
| | GGCCGTTTACTACTGTGCTGCGGTCGAC |
| AD9 gBlock 2 | CTTCTAAGTGGACATGAGTGATTGGATCAAGACGTTTGCAAAAGGGACTA |
| (SEQ ID 079) | GGCTCATAGTAACTTCGCCTGGTAAGTAATTTTTTTTCTGTTTTTATTCCAGT |
| | AATGAAAAACTGACTTCTAAGTGGACATGCTGATGGCGCGAGGGAGGC |
| AD9 Bridge | CTGTGCTGCGGTCGACNNNNNNNNNNNNCTTCTAAGTGGACATGAGTG |
| (SEQ ID 080) | ATTGG |
| AD9 Library | GCCTTGCCAGCCCGCTCAGCTTCTAAGTGGACATGTGGAGCAGTTCCAGCT |
| (SEQ ID 081) | ATCCATTTCCACGGAAGTCAAGAAAAGTATTGACATACCTTGCAAGATATC |
| | GAGCACAAGGTTTGAAACAGATGTCATTCACTGGTACCGGCAGAAACCAA |
| | ATCAGGCTTTGGAGCACCTGATCTATATTGTCTCAACAAAATCCGCAGCTC |
| | GACGCAGCATGGGTAAGACAAGCAACAAAGTGGAGGCAAGAAAGAATTC |
| | TCAAACTCTCACTTCAATCCTTACCATCAAGTCCGTAGAGAAAGAAGACAT |
| | GGCCGTTTACTACTGTGCTGCGGTCGACNNNNNNNNNNNNCTTCTAAGT |
| | GGACATGAGTGATTGGATCAAGACGTTTGCAAAAGGGACTAGGCTCATAG |
| | TAACTTCGCCTGGTAAGTAATTTTTTTTCTGTTTTTATTCCAGTAATGAAAA |
| | ACTGACTTCTAAGTGGACATGCTGATGGCGCGAGGGAGGC |

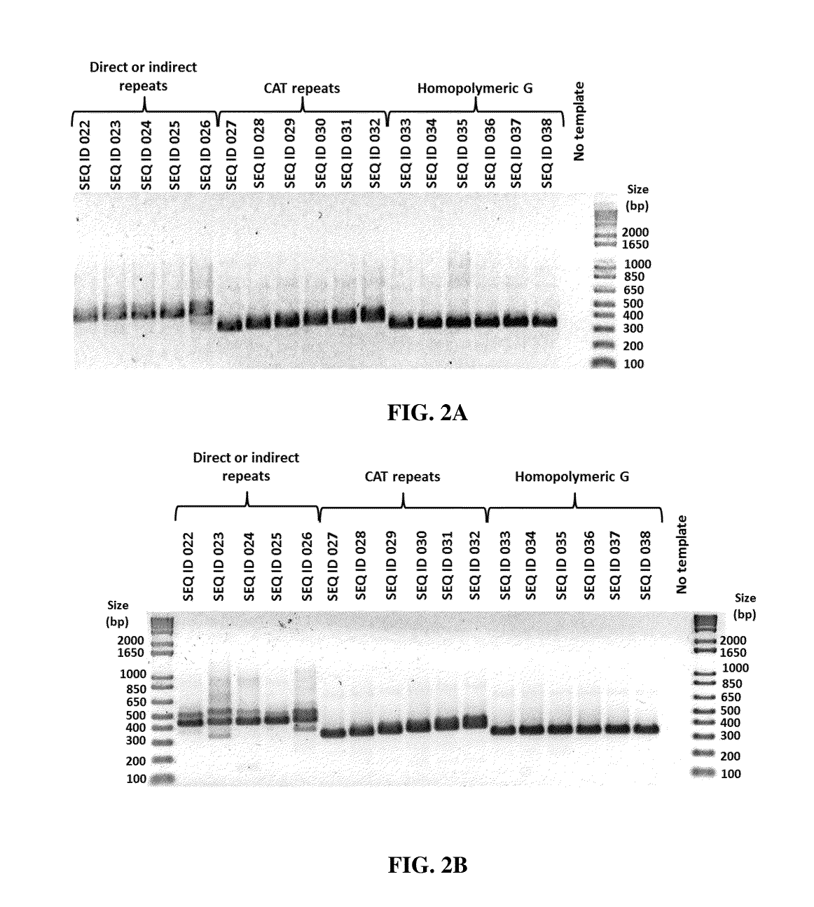

The assembled products were purified using Agencourt AMPure XP magnetic beads (Beckman Coulter) at a bead:PCR volume ratio of 0.8:1, following manufacturer recommended conditions for washing and drying. The DNA was eluted using 45 μl of nuclease-free water and 5 μl of eluted DNA was added as the template into a second PCR reaction with the primers and the same PCR conditions used previously for assembly. These re-amplified PCR products were purified using AMPure XP magnetic beads as described previously and separated on a 2% agarose gel, stained with GelRed nucleic acid gel stain (Biotium), and visualized on a UV transilluminator. All of the re-amplified assemblies resulted in a single band of the expected size (FIG. 2A).

Error correction is an optional step that serves to decrease the number of mutations in the final construct. This was performed by first heating 100 ng of re-amplified assembly product in 20 ul of 1×HF buffer (New England Biolabs) to 95° C. and cooling slowly to form heteroduplex DNA where mutations are present. The heteroduplex DNA was treated with 1 μl Surveyor® Nuclease S (Integrated DNA Technologies) and 0.0125 units of exonuclease III (New England Biolabs) in 1×HF buffer and a final volume of 25 μl. The reaction was incubated at 42° C. for 1 hour.

After incubation, 5 μl of the error correction reaction was added as template in a PCR reaction using the same primers and reaction conditions as in the previous reactions. The post-error correction products were purified using AMPure XP magnetic beads using a bead:DNA volume ratio of 1:1 and separated on a 2% agarose gel and visualized as stated previously. All lanes contained the band of the expected size (FIG. 2B).

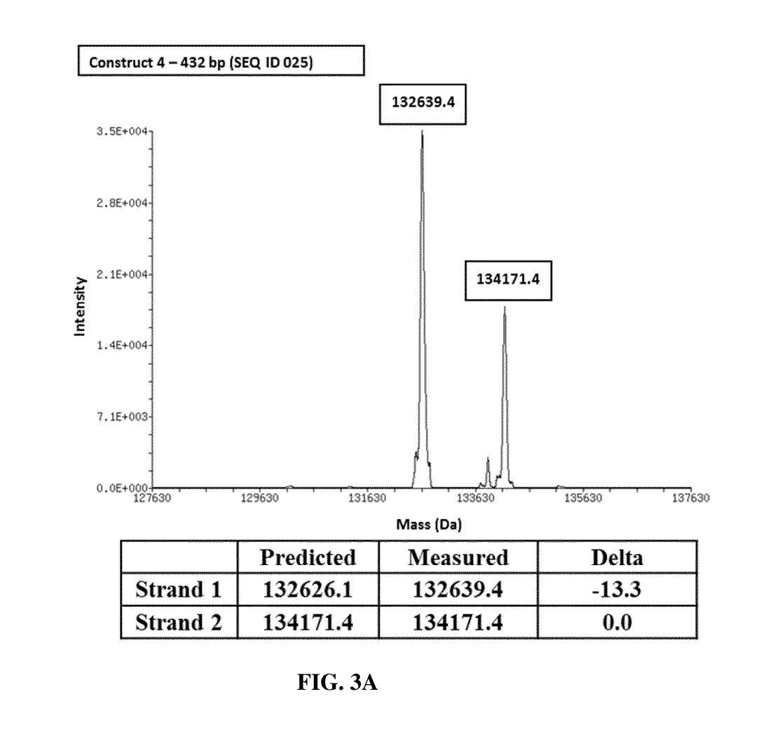

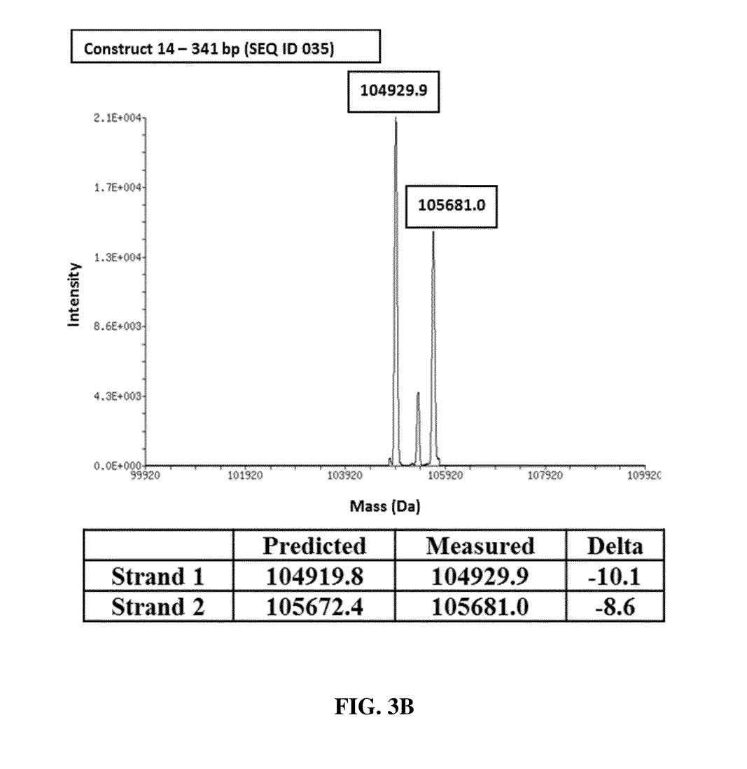

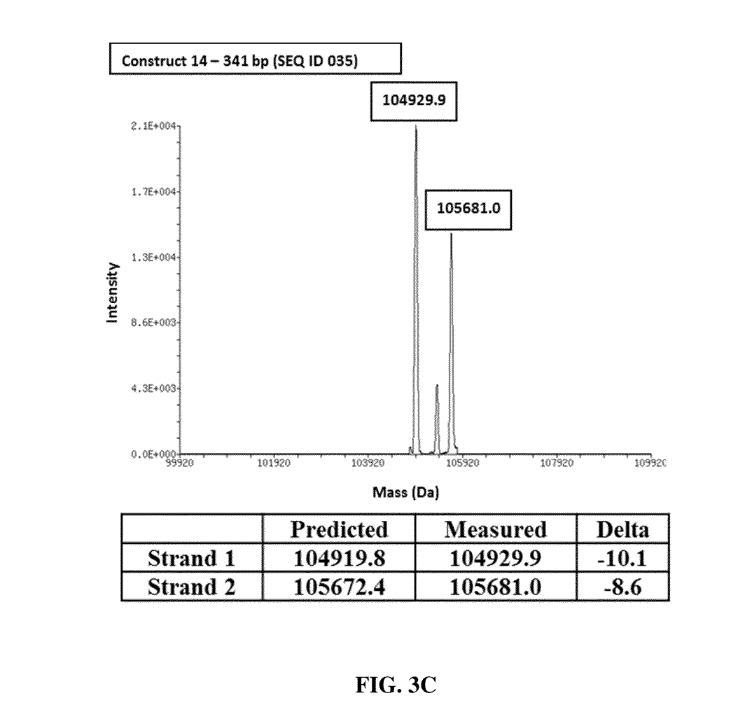

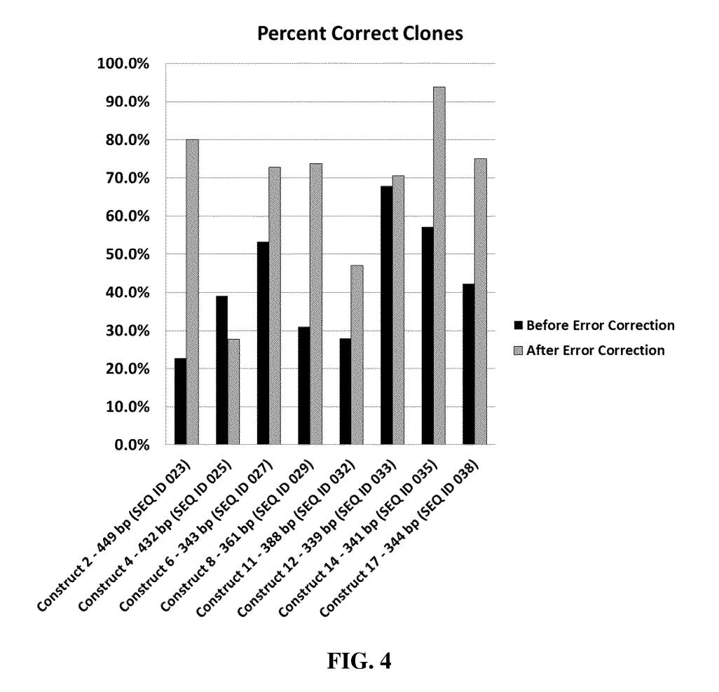

One pmole of each post-error correction product was subjected to Electrospray Mass Spectroscopy (ESI) analysis. The expected mass for each strand was obtained for all desired sequences and was the most prevalent species. Three examples are shown (FIG. 3A-C). In addition, selected products before and after error correction were cloned and sequenced using BigDye® Terminator v3.1 Cycle Sequencing Kit and a 3730×1 DNA Analyzer (Life Technologies). Between 15 and 30 clones had good quality full sequencing coverage and were used to determine the percent of correct clones (FIG. 4). While error correction increased the number of perfect clones, a significant number of correct clones were obtained even in the absence of error correction.

### Example 2

This example demonstrates the incorporation of 3 degenerate bases into a double stranded sequence through the use of a bridging oligonucleotide and double stranded DNA fragments to create a library of 32 DNA sequence variants. This type of library is useful for making single amino acid replacement libraries.

A double stranded DNA library containing a fixed region of degeneracy was created by incorporating NNK (N is the IUB code for A, G, C, T and K is the code for G or T) mixed base sites into the bridge sequence and assembling the bridging oligonucleotide between two double stranded DNA fragments. In this example the assembly was done using two gBlocks containing Illumina TruSeq P5 and P7 adapter sequences, which allowed for next generation sequencing analysis of the prevalence of mixed bases at each position in the final library.

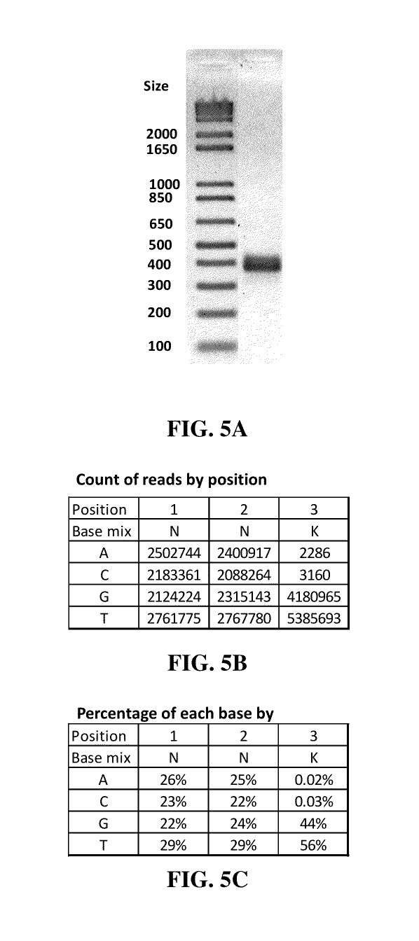

P5 gBlock 1 (SEQ ID NO: 39) and P7AD002 gBlock 2 (SEQ ID NO: 40) were combined with the 1NNK bridge (SEQ ID NO: 41), which contained an internal NNK degenerate sequence flanked by 18 bases of sequence overlapping with each gBlock. The assembly PCR reaction contained equimolar 250 fmoles of each gBlock and bridging oligonucleotide, 200 nM primers (SEQ ID NO: 42 and 43), 0.02 U/μL of KOD Hot Start DNA polymerase, 1×KOD Buffer, 0.8 mM dNTPs and 1.5 mM MgSO₄ in a 50 μl final volume. PCR cycling was performed using the following settings: (95^{3:00}−(95^{0:20}−61^{0:10}−70^{0:20})×25 cycles. This resulted in the construction of the 1NNK gBlock library (SEQ ID NO: 44) with a complexity of 32 variants (4²*2¹=32) and represents codons encoding all 20 standard amino acids and the stop codon TAG. The library was purified using AMPure XP magnetic beads at a bead:DNA volume ratio of 0.8:1, separated on a 2% agarose gel, and visualized as described in Example 1. A single band at the expected 355 base pair size was observed (FIG. 5A).

The 1NNK gBlock library was subjected to next-generation sequencing analysis on an Illumina MiSeq platform with a read length of 250×250 cycles. By only using overlapping paired end reads, the perfectly matched reads were used to determine the sequence and drastically lower the error rate from the sequencer. FIG. 5B shows the count of reads for each degenerate position, and FIG. 5C illustrates the base distribution in percentages. For the N base positions, all four nucleotides were present in an approximately even distribution centering around 25% (22 to 29%). For the K base position, the two nucleotides were present close to the expected 50% prevalence for the G and T nucleotides (44 and 56%, respectively). A very low percentage of the nucleotides at the K base position were the A or C nucleotides (0.02% or 0.03%, respectively).

### Example 3

This example demonstrates the contiguous incorporation of 18 degenerate bases into a double stranded sequence through the use of a bridging oligonucleotide and double stranded DNA fragments to create a library with more than 1 billion sequence variants. This type of library is useful for consecutive amino acid replacements.

A double stranded DNA library containing a highly complex region of degeneracy was created by assembling between two double stranded fragments a bridging oligonucleotide containing 6 tandem NNK degenerate regions. This allows the construction of a high complexity library [(4²*2¹)⁶=1,073,741,824 variants]. The gBlock library was assembled using P5 gBlock 1 (SEQ ID NO: 39), P7AD009 gBlock 2 (SEQ ID NO: 45), 6NNK Bridge (SEQ ID NO: 46) and primers (SEQ ID NO: 42 and 43) under the same PCR conditions and purification described in example 2. This resulted in the construction of the 6NNK gBlock library (SEQ ID NO: 47).

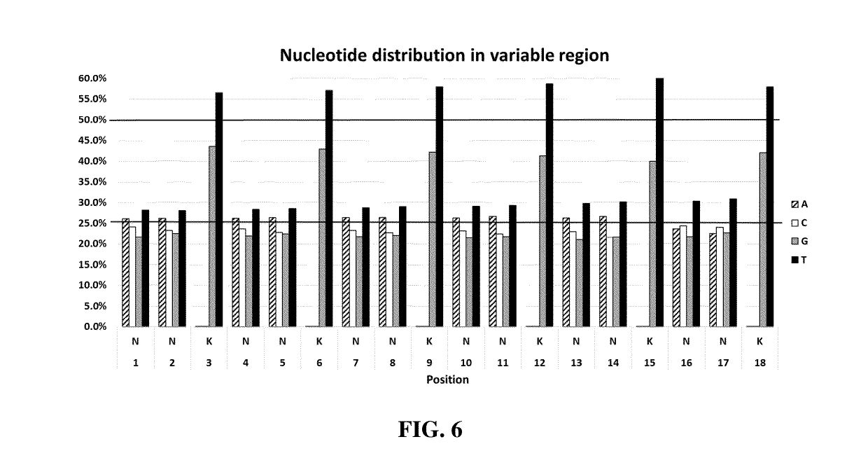

The high complexity 6NNK gBlock library was subjected to next generation sequencing analysis on an Illumina MiSeq platform with a read length of 250×250 cycles. FIG. 6 shows the nucleotide distribution at each position in the variable region of the library. For the N base positions, all four nucleotides were present in an approximately even distribution centering around the theoretical 25% mark. For the K base positions, the two nucleotides were present at approximately the theoretical 50% mark for the G and T nucleotides, however it was observed that T was slightly more prevalent than expected at all positions in this example.

### Example 4

This example demonstrates the incorporation of non-contiguous degenerate base positions into a double stranded sequence through the use of a bridging oligonucleotide and double stranded DNA fragments. This type of library is useful for introducing discrete islands of amino acid changes in between fixed sequence regions.

A double stranded DNA library containing non-contiguous degenerate base regions was created by assembling between two double stranded DNA fragments a bridging oligonucleotide containing one region of NNKNNK and two single NNK regions separated by 6 or 9 fixed DNA bases. GFP-A gBlock 1 (SEQ ID 048) and GFP-A gBlock 2 (SEQ ID 049) were combined with GFP-A Bridge (SEQ ID 050), which contained the regions of degeneracy flanked by overlap with each gBlock. The assembly PCR reaction contained equimolar 250 fmoles of each gBlock and bridging oligonucleotide, 200 nM primers (SEQ ID 051 and 052), 0.02 U/μL of KOD Hot Start DNA polymerase, 1×KOD Buffer, 0.8 mM dNTPs and 1.5 mM MgSO₄ in a 50 μl final volume. PCR cycling was performed using the following settings: (95^{3:00}−(95^{0:20}−65^{0:10}−70^{0:20})×25 cycles. This resulted in the construction of the GFP-A 444 bp library (SEQ ID 053).

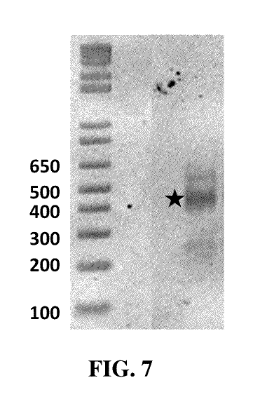

The assembled library was diluted 100-fold in water and re-amplified (optional step) with just the terminal primers under the same PCR reaction and cycling conditions. The re-amplified library was separated on a 2% agarose gel and visualized as described in example 1. The full length product is 444 bp, and is indicated by a black star in FIG. 7.

### Example 5

This example demonstrates the creation of a library in which multiple bridging oligonucleotides, each containing a degenerate region at successive positions, are pooled and assembled with double stranded DNA fragments to form a double stranded DNA walking library. This type of library is useful for introducing one amino acid change at a time along the sequence of interest, while keeping the other amino acids constant.

An example of the construction of a double stranded DNA library containing degenerate regions at successive positions along the sequence, while keeping the rest of the sequence constant, is illustrated in FIG. 8A. This can be referred to as a walking library. Multiple bridging oligonucleotides are designed to contain consecutive NNK degenerate bases walking along the region of interest in the bridge sequence. All bridging nucleotides in the pool share the same regions of gBlock overlap for assembly. In this example, 10 bridging oligonucleotides were pooled by combining equimolar amounts of each bridge (Seq ID 056-065). The pool was diluted to 5 nM each bridge (50 nM total pool) and 250 fmoles of bridge pool was combined with 250 fmoles of each gBlock (Seq ID 054 and 055). The mixture was cycled at 95^{3:00}−(95^{0:20}−60^{0:10}−70^{0:20})×25 cycles using 200 nM primers (Seq ID 066 and 067), 0.02 U/uL of KOD Hot Start DNA polymerase, 1×KOD buffer, 0.8 mM dNTP and 1.5 mM MgSO₄ in a 50 μl final volume.

The gBlock walking library product was purified with AMPure XP beads at a bead:DNA volume ratio of 0.8:1 and eluted in 25 μl water, followed by 100-fold dilution in water. The library was re-amplified (optional step) using 5 μl of the diluted library, 200 nM primers, and using the same PCR reaction conditions as in the previous step but with only 10 cycles of PCR. The libraries before and after 10 cycles of re-amplification were separated on a 2% agarose gel and visualized as described in example 1. The full length 408 bp product is present with or without re-amplification (FIG. 8B).

### Example 6

This example illustrates the detrimental effect of subjecting a double stranded DNA library containing a variable region to extensive PCR cycling during re-amplification.

Three different libraries were constructed using two gBlocks and one bridging oligonucleotide for each library assembly. The AD7 library (SEQ ID 073) was constructed using AD7 gBlock 1, AD7 gBlock 2, and AD7 Bridge (SEQ ID 070-072). The AD8 library (SEQ ID 077) was constructed using AD8 gBlock 1, AD8 gBlock 2, and AD8 Bridge (SEQ ID 074-076). The AD9 library (SEQ ID 081) was constructed using AD9 gBlock 1, AD9 gBlock 2, and AD9 Bridge (SEQ ID 078-080). The bridging oligonucleotide in each library contained 12 contiguous N mixed bases (equal mix of A, T, G, and C at each position) flanked by a region of overlap with each gBlock.

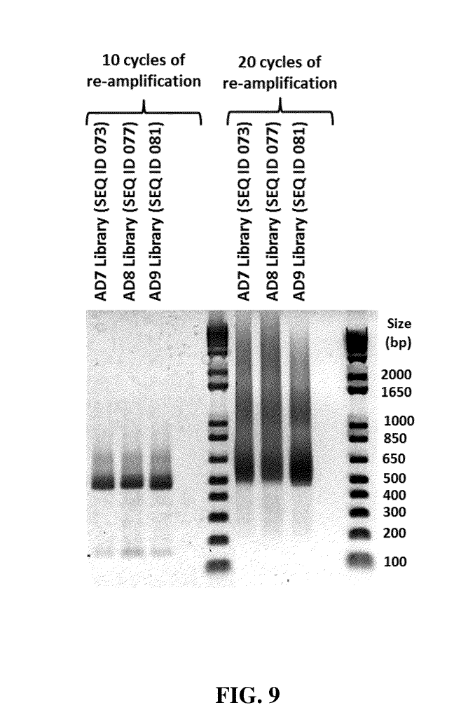

The library was assembled by combining equimolar amounts, 250 fmoles of gBlock1, gBlock 2, and bridging oligonucleotide for each library. The mixture was cycled at 95° C.^{3:00} (95° C.^{0:20}+64° C.^{0:10}+70^{0:20})×25 cycles using 200 nM primers (Seq ID 068 and 069), 0.02 U/uL of KOD Hot Start DNA polymerase, 1×KOD buffer, 0.8 mM dNTP and 1.5 mM MgSO₄ in a 50 μl final volume. The library product was purified with AMPure XP magnetic beads at a bead:DNA volume ratio of 0.8:1 and eluted in 45 μl water, followed by 100-fold dilution in nuclease-free water. Each library was re-amplified using 5 μl of the diluted library, 200 nM primers, and the same PCR reaction conditions as in the previous step but with either 10 or 20 cycles of PCR. The library products after re-amplification were separated on a 2% agarose gel and visualized as described in example 1 (FIG. 9). A band of the expected size of 494 bp is evident after 10 cycles of re-amplification, however 20 cycles of re-amplification results in smeared products in the gel lanes for all 3 libraries. This demonstrates the importance of limiting the number of cycles of re-amplification PCR performed on the constructed library.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of constructing a double stranded DNA fragment or library, said method comprising incorporating sequences between clonal or non-clonal double stranded DNA fragments (gene blocks), the method comprising:
a) forming a mixture comprised of a first gene block, a second gene block, and a bridging oligonucleotide set, said bridging oligonucleotide set comprising one or more bridging oligonucleotides, wherein each bridging oligonucleotide contains a first region that is hybridizable to a portion of the first gene block and a second region that is hybridizable to a portion of the second gene block;
b) subjecting the mixture to reagents and conditions for PCR to assemble the gene blocks and bridge(s) thereby generating and optionally amplifying a double stranded DNA fragment or library, wherein the sequence generated is comprised of the first gene block, a bridge sequence of the bridging oligonucleotide(s), if any, that did not hybridize to a gene block, and the second gene block.

2. The method of claim 1 wherein the first gene block is greater than 50 base pairs and the second gene block is greater than 50 base pairs.

3. The method of claim 1 wherein the mixture further comprises one or more additional gene blocks wherein the one or more bridging oligonucleotides contain one or more regions that are hybridizable to a portion of the one or more additional gene blocks.

4. The method of claim 1 wherein the mixture further comprises one or more additional gene blocks and one or more additional bridging oligonucleotides wherein the one or more additional bridging oligonucleotides contains (i) a region hybridizable to an additional gene block, and (ii) a region hybridizable to another additional gene block, the first gene block or the second gene block.

5. The method of claim 1 wherein the mixture is assembled and amplified less than twenty PCR cycles.

6. The method of claim 1 wherein the mixture is assembled and amplified between 5 and 15 PCR cycles.

7. The method of claim 1 wherein the bridging oligonucleotide set is comprised of bridging oligonucleotides containing at least one degenerate base.

8. The method of claim 1 wherein the bridging oligonucleotide set is comprised of bridging oligonucleotides containing from 1-30 degenerate bases.

9. The method of claim 1 wherein the bridging oligonucleotide set contains at least one mismatch or non-standard base located within the first region or second region.

10. The method of claim 1 wherein the bridging oligonucleotide set contains fixed regions of low complexity, direct or indirect repeats, and/or homopolymeric nucleotide runs.

11. The method of claim 1 wherein the bridging oligonucleotide set consists of a sequence that is hybridizable to the first gene block and sequence that is hybridizable to a second gene block, and upon assembly does not add an additional sequence between the first and second gene blocks.

12. The method of claim 1 wherein the bridging oligonucleotide set is comprised of bridging oligonucleotides wherein the first hybridizable region is between 10-50 bases and the second hybridizable region is between 10-50 bases.

13. The method of claim 1 wherein the bridging oligonucleotide set comprises two or more bridging oligonucleotides with an identical sequence except for mixed base site locations varying along the bridge sequence of the bridging oligonucleotide(s) that did not hybridize to a gene block.

14. The method of claim 1 wherein the bridging oligonucleotide set contains non-random nucleotide variation at specific location(s).

15. The method of claim 14 wherein the non-random variation at specific locations is for targeted codon changes.

16. The method of claim 1 wherein the bridging oligonucleotide set contains a region of low complexity or repeating elements.

17. The method of claim 1 wherein the mixed base molar ratios in a variable region of a bridging oligonucleotide set is controlled by hand mixing phosphoramidites at the desired ratio.

18. A method of constructing a double stranded DNA fragment or library, said method comprising incorporating sequences between clonal or non-clonal double stranded DNA fragments (gene blocks), the method comprising:
a) forming a mixture comprised of more than two gene blocks, and a bridging oligonucleotide set, said bridging oligonucleotide set comprising one or more bridging oligonucleotides, and wherein each bridging oligonucleotide contains a first region that is hybridizable to a portion of one gene block and a second region that is hybridizable to a portion of another gene block wherein, when mixed together, a resulting product comprises successive gene blocks linked by bridging oligonucleotides;
b) subjecting the mixture to reagents and conditions for PCR to assemble the gene blocks and bridge(s) and thereby generating and amplifying a double stranded DNA fragment or library, wherein the sequence generated is comprised of the first gene block, the bridge sequence of the bridging oligonucleotide(s), and the second gene block.

19. A kit for the manufacture of a double-stranded DNA fragment library, said kit comprising:
(a) two or more gene blocks; and
(b) one or more bridging oligonucleotide, wherein each bridging oligonucleotide contains a first region of 10-50 bases substantially complementary to a strand of a first gene block and a second region of 10-50 bases substantially complementary to a strand of a second gene block, and wherein the bridging oligonucleotide contains 1-30 degenerate bases.

20. The kit of claim 20 wherein each gene block is greater than 50 base pairs.

21. The kit of claim 19 further comprising multiple bridging oligonucleotides containing varying regions of degenerate bases.
